(19) United States
(12) Patent Application Publication
(10) Pub. No.: US 2009/0208471 A1
(43) Pub. Date: Aug. 20, 2009

(54) Isolation and Use of Human Regulatory T Cells

(76) Inventor: Theodore J. Yun, San Marcos, CA (US)

Correspondence Address: STERNE, KESSLER, GOLDSTEIN & FOX, P.L.L.C., 1100 NEW YORK AVE., N.W., WASHINGTON, DC, 20005, US

(21) Appl. No.: 12/296,386

(22) PCT Filed: Apr. 6, 2007
(86) PCT No.: PCT/US07/08581

§ 371 Date: Mar. 27, 2009

Related U.S. Application Data
(60) Provisional application No. 60/789,918, filed on Apr. 7, 2006.

(51) Int. Cl. A61K 35/12; C12N 5/08; C12Q 1/02; A61P 37/06

(52) U.S. Cl. 424/93.71; 435/372.3; 435/29

(57) ABSTRACT

The present invention provides a new method for isolating and enriching human regulatory T cells. The enriched cells are useful in the treatment of autoimmune disease.

### BACKGROUND OF THE INVENTION

1. Field of the Invention

The present invention is related to methods for isolating regulatory T (Treg) cells, enriched populations of Treg cells, and methods for treating autoimmune disease.

2. Background

Central tolerance is the predominant mechanism responsible for elimination of lymphocytes with self specificities; however, it is not a complete process. Peripheral tolerance mechanisms have been demonstrated to regulate potentially autoreactive lymphocytes. Most of these peripheral tolerance mechanisms are cell-intrinsic and involve clonal deletion, anergy induction or receptor revision (Ali et al., J. Immunol. 171:6290-6296 (2003); McMahan and Fink, J. Immunol. 165:6902-6907 (2000)). Accumulating evidence suggests that a dominant, cell-extrinsic mechanism exists to control activation of autoreactive lymphocytes. In the past decade, CD4⁺CD25⁺ regulatory T (Treg) cells have emerged as a novel paradigm of immune self-tolerance mechanisms (Sakaguchi et al., Ann. Rev. Immunol. 22: 531-562 (2004); Kronenberg and Rudensky, Nature 435:598-604 (2005)). This subset of lymphocytes can exert a suppressive function on their CD4⁺CD25⁻ cohorts (Sakaguchi et al., J. Immunol. 155:1151-1164 (1995)). Furthermore, the regulatory phenotype is dominant in adoptive transfers of enriched CD4⁺CD25⁺ cells that can compensate in an animal genetically lacking or rendered deficient of Treg cells (Fontenot et al., Immunity 22:329-341 (2005); Asano et al. J. Exp. Med. 184:387-396 (1996); Hori et al., Science 299:1057-1061 (2003)).

Treg cell dysfunction has dramatic consequences, and the role of Treg cells has been implicated in several disease models. Diminished numbers of Treg cells or their impaired function may contribute to development of autoimmune disease (Setoguchi et al., J. Exp. Med. 201:723-735 (2005)). In mice experimentally rendered deficient of Treg cells, for example neonatally thymectomized mice (Asano et al, 1996), or genetically deficient for Treg cells, such as the scurfy mutant strain (Ramsdell, F. Immunity 19: 165-168 (2003)), mice develop and succumb to multi-organ autoimmune disease. Adoptive transfer of enriched CD4⁺CD25⁺ cells can prevent mice from developing autoimmune disease and rescue mice that have developed these diseases (Fontenot et al. 2005; Hori et al., 2003). Conversely, increased numbers of Treg cells have been suggested to account for impaired immune surveillance of tumor infiltrating T cells (Curiel, T. J., et al., Nat. Med. 10:942-949 (2004); Ichihara, F. et al. Clin. Cancer Res. 9:4404-4408 (2003); Ko, K. et al. J. Exp. Med. 202:885-891 (2005); Liyanage, U. K. et al. J. Immunol. 169:2756-2761 (2002); Woo, E. Y. et al., Cancer Res. 61:4766-4772 (2001); Woo, E. Y. et al., J. Immunol. 168:4272-4276 (2002); Yu, P. et al., J. Exp. Med. 201:779-791 (2002)).

The existence of Treg cells has been solidified by identification of the FoxP3 transcription factor (Hori et al., 2003; Yasayko, J. E. et al., Nat. Genet. 27:68-73 (2001); Fontenot, J. D. et al., Nat. Immunol. 4:330-336 (2003); Khattri, R. et al., Nat. Immunol. 4:337-342 (2003)). This molecule is a member of the forkhead winged-helix transcription factor family. Currently, no transcriptional targets have been identified, although multiple genes involved in T cell function have consensus forkhead binding sites (Schubert, L. A. et al., J. Biol. Chem. 276:37672-37679 (2001)). FoxP3 overexpression results in the repression of multiple genes. Spontaneous or targeted mutations that result in a null mutation lead to a defect in Treg cell development. Conversely, transgenic or enforced expression of FoxP3 in CD4 T cells can functionally convert these cells into Treg cells that suppress autoreactive T cells in vivo (Hori et al., 2003; Fontenot et al., 2003; Khattri et al., 2003). These loss-of-function and gain-of-function genetic experiments strongly argue for the essential requirement of FoxP3 function in Treg cell development.

Although Treg cells have been characterized in mice, defining this population in humans has been challenging because in vivo experiments are not possible. The primary challenge has been identifying and isolating these cells based on extracellular markers. Since their identification, researchers have been relying on CD25 to positively identify Treg cells. However, CD25 is an activation marker on T cells, and therefore is not exclusive to Treg cells. Also, the degree of heterogeneity present in circulating CD4⁺CD25⁺ cells is controversial. In mice expressing a knock-in FoxP3/GFP fusion protein, the majority of CD4⁺CD25⁺ T cells are FoxP3⁺, and thus are Treg cells (Fontenot et al., 2005; Wan Y. Y. and Flavell, R. A. Proc. Natl. Acad. Sci. USA 102:5126-5131 (2005)).

In contrast, in humans, it has been demonstrated that multiple subpopulations comprise the CD4⁺CD25⁺ T cell population (Baecher-Allan, C. et al., J. Immunol. 167:1245-1253 (2001); Jonuleit, H. et al., J. Exp. Med. 193:1285-1294 (2001); Taams, L. S. et al., Eur. J. Immunol. 32:1621-1630 (2002); Yagi, H. et al., Int. Immunol. 16:1643-1656 (2004)). Regulatory activity was shown to be enriched in CD4⁺CD25^{hi} cells, while CD4⁺CD25^{med} cells were not potent suppressors. Also, using CD4⁺RO or CD45RA to segregate the human CD4⁺CD25⁺ population, regulatory activity was found in the CD45RO (CD45RA⁻) subpopulation. Recently, in synovial fluid infiltrates from rheumatoid arthritis (RA) patients, it was found that regulatory activity was enriched in CD4⁺CD25⁺CD27⁺ cells (Ruprecht, C. R. et al., J. Exp. Med. 201:1793-1803 (2005)). However, isolating human Treg cells from peripheral blood mononuclear cells (PBMCs) using any of these criteria alone or in combination has met technical limitations. Using CD25 levels to sort Treg cells by fluorescence activated cell sorting (FACS) is difficult because fluorescent intensity is arbitrary, and hence imprecise. Also, the expression of CD45RO (or CD45RA) does not segregate the population into CD25^{hi} and CD25^{lo}. In PBMCs as opposed to inflammatory infiltrates, CD27 is expressed by the majority of CD4⁺ cells, and does not define a subpopulation of CD4⁺CD25⁺ cells. Thus, the surface phenotype of human Treg cells remains to be resolved.

### BRIEF SUMMARY OF THE INVENTION

A new method for isolating and enriching human Treg cells is provided. The enriched cells are useful in the treatment of autoimmune disease by acting to suppress self-reactive immune cells.

In one embodiment, the invention provides an isolated population of regulatory T (Treg) cells wherein the population comprises at least 75% regulatory T cells and less than 25% non-regulatory T cells, wherein the regulatory T cells express CD4 and CD25; and do not express detectable levels CD45RA and CD127. The isolated population of Treg cells may also express FoxP3.

The isolated population of Treg cells can be isolated from peripheral blood mononuclear cells, synovial fluid and from tissue. In some embodiments, the tissue is selected from the group consisting of: spleen, thymus, lymph nodes, bone marrow, Peyer's patches and tonsils.

In another embodiment, the invention is directed to an enriched population of regulatory T cells, wherein the cells express CD4 and CD25; and do not express detectable levels of CD45RA and CD127. The enriched population of cells can be enriched from a population of peripheral blood mononuclear cells, synovial fluid or from a tissue sample. In one embodiment, the population of Treg cells is enriched at least 2-fold. In another embodiment, the population of Treg cells is enriched at least 5-fold. In a further embodiment, the population of Treg cells is enriched at least 10-fold. In a further embodiment, the population of Treg cells is enriched at least 50-fold.

The invention is also directed to a method of enriching a population of regulatory T cells, comprising: (a) contacting a population of cells with a first, second, third and fourth reagent, which respectively bind CD4, CD25, CD45RA and CD127; and (b) selecting cells that bind to the first and second reagent and do not bind to the third or fourth reagent, wherein the selected cells are enriched for regulatory T cells. In one embodiment, the first, second, third and fourth reagents comprise antibodies that respectively bind CD4, CD25, CD45RA and CD127. The antibodies may be conjugated to a fluorochrome or magnetic particle. In a further embodiment, the cell selection is performed by flow cytometry, fluorescence activated cell sorting, magnetic selection, affinity chromatography or panning, or combinations thereof.

The invention is also directed to a method of enriching a population of regulatory T cells, comprising: (a) contacting a population of cells with a first, second, third and fourth reagent, which respectively bind CD4, CD25, CD45RO and CD127; and (b) selecting cells that bind to the first, second and third reagent and do not bind to the fourth reagent, wherein the selected cells are enriched for regulatory T cells. In one embodiment, the first, second, third and fourth reagents comprise antibodies that respectively bind CD4, CD25, CD45RO and CD127. The antibodies may be conjugated to a fluorochrome or magnetic particle. In a further embodiment, the cell selection is performed by flow cytometry, fluorescence activated cell sorting, magnetic selection, affinity chromatography or panning, or combinations thereof.

The invention is also directed to a method of enriching a population of regulatory T cells comprising: (a) contacting a population of cells with antibodies that bind CD4 and CD25; (b) retaining cells that bind to said antibodies that bind CD4 and CD25; (c) contacting said retained cells with antibodies that bind CD45RA and CD127; and (d) retaining cells that do not bind to said antibodies that bind CD45RA and CD127, wherein said retained cells are enriched for regulatory T cells. In one embodiment, the population of cells are peripheral blood mononuclear cells or synovial fluid cells. In another embodiment, the population of cells are from a tissue selected from the group consisting of: spleen, thymus, lymph nodes, bone marrow, Peyer's patches, and tonsils.

In a further embodiment, the antibodies are conjugated to a fluorochrome or magnetic particle and the retaining step is performed by flow cytometry, fluorescence activated cell sorting, magnetic selection, affinity chromatography or panning, or combinations thereof. In another embodiment, the method comprises isolating the enriched population of regulatory T cells. In a further embodiment, the invention is directed to an enriched population of regulatory T cells isolated by the method described above.

The invention is also directed to a method of enriching a population of regulatory T cells comprising: (a) contacting a population of cells with antibodies that bind CD4, CD25 and CD45RO; (b) retaining cells that bind to said antibodies that bind CD4, CD25 and CD45RO; (c) contacting said retained cells with antibodies that bind CD127; and (d) retaining cells that do not bind to said antibodies that bind CD127, wherein said retained cells are enriched for regulatory T cells. In one embodiment, the population of cells are peripheral blood mononuclear cells or synovial fluid cells. In another embodiment, the population of cells are from a tissue selected from the group consisting of: spleen, thymus, lymph nodes, bone marrow, Peyer's patches, and tonsils.

In a further embodiment, the antibodies are conjugated to a fluorochrome or magnetic particle and the retaining step is performed by flow cytometry, fluorescence activated cell sorting, magnetic selection, affinity chromatography or panning, or combinations thereof. In another embodiment, the method comprises isolating the enriched population of regulatory T cells. In a further embodiment, the invention is directed to an enriched population of regulatory T cells isolated by the method described above.

The invention is also directed to a method of suppressing an autoimmune response in a subject comprising: (a) obtaining an enriched population of regulatory T cells, wherein the cells are obtained by: (i) contacting a population of cells with a first, second, third and fourth reagent, which respectively bind CD4, CD25, CD45RA and CD127; and (ii) selecting cells that bind to the first and second reagent and do not bind to the third or fourth reagent, wherein the selected cells are enriched for regulatory T cells; and (b) introducing the enriched population of cells into the subject to suppress the autoimmune response. In one embodiment, the autoimmune response is associated with an autoimmune disease selected from the group consisting of: lupus erythematosus, pemphigus vulgaris, thyreoiditis, thrombocytopenic purpura, Graves disease, diabetes mellitus, myasthenia gravis, Addison's disease, rheumatoid arthritis, multiple sclerosis, psoriasis, uveitis, and autoimmune hemolytic anemia.

In one embodiment, the enriched population of cells is obtained from the subject in need of treatment. In a further embodiment, the population of cells is enriched from peripheral blood mononuclear cells or synovial fluid. In another embodiment, the population of cells is enriched from a tissue sample.

The invention is also directed to a method of suppressing an autoimmune response in a subject comprising: (a) obtaining an enriched population of regulatory T cells, wherein the cells are obtained by: (i) contacting a population of cells with a first, second, third and fourth reagent, which respectively bind CD4, CD25, CD45RO and CD127; and (ii) selecting cells that bind to the first, second and third reagent and do not bind to the fourth reagent, wherein the selected cells are enriched for regulatory T cells; and (b) introducing the enriched population of cells into the subject to suppress the autoimmune response. In one embodiment, the autoimmune response is associated with an autoimmune disease selected from the group consisting of: lupus erythematosus, pemphigus vulgaris, thyreoiditis, thrombocytopenic purpura, Graves disease, diabetes mellitus, myasthenia gravis, Addison's disease, rheumatoid arthritis, multiple sclerosis, psoriasis, uveitis, and autoimmune hemolytic anemia.

In one embodiment, the enriched population of cells is obtained from the subject in need of treatment. In a further embodiment, the population of cells is enriched from peripheral blood mononuclear cells or synovial fluid. In another embodiment, the population of cells is enriched from a tissue sample.

The invention is also directed to a method of enriching a population of regulatory T cells, comprising: (a) contacting a population of cells with a first reagent or reagents which binds to a group of markers on non-CD4⁺ immune cells; and a second, third and fourth reagent, which respectively bind CD25, CD45RA and CD127; and (b) selecting cells that bind to the second reagent and do not bind to the first, third or fourth reagent, wherein the selected cells are enriched for regulatory T cells. In one embodiment, the non-CD4⁺ immune cells are selected from the group consisting of one or more of: cytotoxic T cells, γ/δ T cells, B cells, natural killer cells, dendritic cells, monocytes, granulocytes and erythroid cells. The non-CD4⁺ immune cells comprise cell surface markers selected from the group consisting of one or more of: CD8, CD14, CD16, CD19, CD36, CD56, CD123, TCR γ/δ and glycophorin A.

In one embodiment, the first, second, third and fourth reagents comprise antibodies that respectively bind a group of markers on non-CD4⁺ immune cells, CD25, CD45RA and CD127. In a further embodiment, the antibodies are conjugated to a fluorochrome or magnetic particle. In another embodiment, the cell selection is performed by flow cytometry, fluorescence activated cell sorting, magnetic selection, affinity chromatography or panning, or combinations thereof.

The invention is also directed to a method of enriching a population of regulatory T cells, comprising: (a) contacting a population of cells with a first reagent or reagents which binds to one or more of a group of markers on non-CD4⁺ immune cells; and a second, third and fourth reagent, which respectively bind CD25, CD45RO and CD127; and (b) selecting cells that bind to the second and third reagent and do not bind to the first or fourth reagent, wherein the selected cells are enriched for regulatory T cells. In one embodiment, the non-CD4⁺ immune cells are selected from the group consisting of one or more of: cytotoxic T cells, γ/ε T cells, B cells, natural killer cells, dendritic cells, monocytes, granulocytes and erythroid cells. The non-CD4⁺ immune cells comprise cell surface markers selected from the group consisting of one or more of: CD8, CD14, CD16, CD19, CD36, CD56, CD123, TCR γ/δ and glycophorin A.

In one embodiment, the first, second, third and fourth reagents comprise antibodies that respectively bind a group of markers on non-CD4⁺ immune cells, CD25, CD45RO and CD127. In a further embodiment, the antibodies are conjugated to a fluorochrome or magnetic particle. In another embodiment, the cell selection is performed by flow cytometry, fluorescence activated cell sorting, magnetic selection, affinity chromatography or panning, or combinations thereof.

The invention is also directed to a method of enriching a population of regulatory T cells comprising: (a) contacting a population of cells with antibodies that bind to one or more of a group of markers on non-CD4⁺ immune cells; (b) retaining cells that do not bind to said antibodies that bind to one or more of a group of markers on non-CD4⁺ immune cells; (c) contacting the retained cells with antibodies that bind CD25; (d) retaining cells that bind to said antibodies that bind CD25; (e) contacting the retained cells with antibodies that bind CD45RA and CD127; and (f) retaining cells that do not bind to the antibodies that bind CD45RA and CD127, wherein the retained cells are enriched for regulatory T cells. In one embodiment, the non-CD4⁺ immune cells are selected from the group consisting of one or more of: cytotoxic T cells, γ/δ T cells, B cells, natural killer cells, dendritic cells, monocytes, granulocytes and erythroid cells. The non-CD4⁺ immune cells comprise cell surface markers selected from the group consisting of one or more of: CD8, CD14, CD16, CD19, CD36, CD56, CD123, TCR δ/δ and glycophorin A.

In one embodiment, the population of cells are peripheral blood mononuclear cells or synovial fluid cells. In another embodiment, the population of cells are from a tissue selected from the group consisting of: spleen, thymus, lymph nodes, bone marrow, Peyer's patches, and tonsils.

In a further embodiment, the antibodies are conjugated to a fluorochrome or magnetic particle and the retaining step is performed by flow cytometry, fluorescence activated cell sorting, magnetic selection, affinity chromatography or panning, or combinations thereof. In a further embodiment, the enriched population of regulatory T cells are isolated. The invention is also directed to an enriched population of regulatory T cells isolated by the method described above.

The invention is also directed to a method of enriching a population of regulatory T cells comprising: (a) contacting a population of cells with antibodies that bind to one or more of a group of markers on non-CD4⁺ immune cells; (b) retaining cells that do not bind to said antibodies that bind to one or more or a group of markers on non-CD4⁺ immune cells; (c) contacting the retained cells with antibodies that bind CD25; (d) retaining cells that bind to said antibodies that bind CD25; (e) contacting said retained cells with antibodies that bind CD45RO; (f) retaining cells that bind to said antibodies that bind CD45RO; (g) contacting the retained cells with antibodies that bind CD127; (h) retaining cells that do not bind to said antibodies that bind CD127; wherein said retained cells are enriched for regulatory T cells. In one embodiment, the non-CD4⁺ immune cells are selected from the group consisting of one or more of: cytotoxic T cells, γ/δ T cells, B cells, natural killer cells, dendritic cells, monocytes, granulocytes and erythroid cells. The non-CD4⁺ immune cells comprise cell surface markers selected from the group consisting of one or more of: CD8, CD14, CD16, CD19, CD36, CD56, CD123, TCR γ/δ and glycophorin A.

In one embodiment, the population of cells are peripheral blood mononuclear cells or synovial fluid cells. In another embodiment, the population of cells are from a tissue selected from the group consisting of: spleen, thymus, lymph nodes, bone marrow, Peyer's patches, and tonsils.

In a further embodiment, the antibodies are conjugated to a fluorochrome or magnetic particle and the retaining step is performed by flow cytometry, fluorescence activated cell sorting, magnetic selection, affinity chromatography or panning, or combinations thereof. In a further embodiment, the enriched population of regulatory T cells are isolated. The invention is also directed to an enriched population of regulatory T cells isolated by the method described above.

The invention is also directed to a method of suppressing an autoimmune response in a subject comprising: (a) obtaining an enriched population of regulatory T cells, wherein the cells are obtained by: (i) contacting a population of cells with a first reagent or reagents which binds to one or more of a group of markers on non-CD4⁺ immune cells; and a second, third and fourth reagent which respectively bind CD25, CD45RA and CD127; and (ii) selecting cells that bind to the second reagent and do not bind to the first, third or fourth reagent, wherein the selected cells are enriched for regulatory T cells; and (b) introducing the enriched population of cells into the subject to suppress the autoimmune response. In one embodiment, the non-CD4⁺ immune cells are selected from the group consisting of one or more of: cytotoxic T cells, γ/δ T cells, B cells, natural killer cells, dendritic cells, monocytes, granulocytes and erythroid cells. The non-CD4⁺ immune cells comprise cell surface markers selected from the group consisting of one or more of: CD8, CD14, CD16, CD19, CD36, CD56, CD123, TCR δ/δ and glycophorin A.

In one embodiment, the enriched population of cells is obtained from the subject in need of treatment. In a further embodiment, the population of cells is enriched from peripheral blood mononuclear cells or synovial fluid. In another embodiment, the population of cells is enriched from a tissue sample. In one embodiment, the autoimmune response is associated with an autoimmune disease selected from the group consisting of: lupus erythematosus, pemphigus vulgaris, thyreoiditis, thrombocytopenic purpura, Graves disease, diabetes mellitus, myasthenia gravis, Addison's disease, rheumatoid arthritis, multiple sclerosis, psoriasis, uveitis, and autoimmune hemolytic anemia.

The invention is also directed to a method of suppressing an autoimmune response in a subject comprising: (a) obtaining an enriched population of regulatory T cells, wherein the cells are obtained by: (i) contacting a population of cells with a first reagent or reagents which binds to one or more of a group of markers on non-CD4⁺ immune cells; and a second, third and fourth reagent which respectively bind CD25, CD45RO and CD127; and (ii) selecting cells that bind to the second and third reagent and do not bind to the first or fourth reagent, wherein the selected cells are enriched for regulatory T cells; and (b) introducing the enriched population of cells into the subject to suppress the autoimmune response. In one embodiment, the non-CD4⁺ immune cells are selected from the group consisting of one or more of: cytotoxic T cells, δ/δ T cells, B cells, natural killer cells, dendritic cells, monocytes, granulocytes and erythroid cells. The non-CD4⁺ immune cells comprise cell surface markers selected from the group consisting of one or more of: CD8, CD14, CD16, CD19, CD36, CD56, CD123, TCR γ/δ and glycophorin A.

In one embodiment, the enriched population of cells is obtained from the subject in need of treatment. In a further embodiment, the population of cells is enriched from peripheral blood mononuclear cells or synovial fluid. In another embodiment, the population of cells is enriched from a tissue sample. In one embodiment, the autoimmune response is associated with an autoimmune disease selected from the group consisting of: lupus erythematosus, pemphigus vulgaris, thyreoiditis, thrombocytopenic purpura, Graves disease, diabetes mellitus, myasthenia gravis, Addison's disease, rheumatoid arthritis, multiple sclerosis, psoriasis, uveitis, and autoimmune hemolytic anemia.

The invention is also directed to a method of diagnosing an autoimmune disease comprising detecting a population of regulatory T cells, wherein the regulatory T cells: (a) express CD4 and CD25; and (b) do not express detectable levels of CD45RA and CD127.

Further embodiments, features, and advantages of the present inventions, as well as the structure and operation of the various embodiments of the present invention, are described in detail below with reference to the accompanying drawings.

### BRIEF DESCRIPTION OF THE DRAWINGS/FIGURES

The accompanying drawings, which are incorporated herein and form a part of the specification, illustrate one or more embodiments of the present invention and, together with the description, further serve to explain the principles of the invention and to enable a person skilled in the pertinent art to make and use the invention.

FIG. 1 shows the segregation of CD4⁺CD25⁺ cells after coculture with immature dendritic cells (iDC). CD4⁺CD25⁺ (right panels) or CD4⁺CD25⁻ cells (left panels) were purified from human blood, and cocultured with allogeneic iDC for three days. On each day, cells were harvested and the expression of CD25 was analyzed by flow cytometry. A gate was established on the live population, and the percentage of CD25^{hi} cells is shown. The relevant isotype control is shown in light trace.

FIG. 2 shows that segregation of CD4⁺CD25⁺ cells is dependent on interaction with CD80 and/or CD86. (a) CD4⁺CD25⁺ cells were purified from human blood, and cocultured with allogeneic iDC (bold histogram) or media alone (light histogram) for three days. CD25 expression was analyzed by flow cytometry. A gate was established on the live population, and the percentage of CD25^{hi} cells is shown. The relevant isotype control is shown in z trace. (b) The SB B-lymphoblastoid cell line is able to mimic the ability of iDC to segregate the CD4⁺CD25⁺ population. CD4⁺CD25⁺ (right panels) or CD4⁺CD25⁻ cells (left panels) were purified from human blood, and cocultured with allogeneic iDC (top panels) or SB cells at a ratio of 1:5 (iDC) or 1:10 (SB cells) for three days. CD25 expression was analyzed by flow cytometry. A gate was established on the live primary lymphocyte population, and the percentage of CD25^{hi} cells is shown. (c) Blocking interaction of T cells with CD80 or CD86 inhibits the ability of SB cells to segregate the CD4⁺CD25⁺ population. CD4⁺CD25⁺ (right panels) or CD4⁺CD25⁻ cells (left panels) were purified from human blood, and cocultured with SB cells at a ratio of 1:10 in the presence of 20 μg soluble CD152-IgFc (bottom panels) or nonspecific monoclonal antibody (top panels) for three days. CD25 expression was analyzed by flow cytometry. A gate was established on the live primary lymphocyte population, and the percentage of CD25^{hi} cells is shown.

FIG. 3 shows that CD4⁺CD25^{+>+} cells are the only subpopulation from the iDC cocultures that can mediate suppression. CD4⁺CD25⁺ or CD4⁺CD25⁻ cells were purified from human blood, and cocultured with allogeneic iDC for three days. Cultures were stained with primary conjugated anti-CD4 and anti-CD25 mAbs, then sorted by FACS into CD4⁺CD25⁻, CD4⁺CD25^{->+}, CD4⁺CD25^{+>-}, CD4⁺CD25^{+>+} subpopulations. Subpopulations were combined with freshly isolated CD4⁺CD25⁻ cells (Tᵣₑₛₚ) at the ratios indicated (Subpopulation to Tᵣₑₛₚ). For stimulators, allogeneic SB cells were used at a ratio of 1:10 SB to Tᵣₑₛₚ. Each culture condition was established in triplicate. (a) Concentrations of the indicated cytokines in each culture are shown. Cytokine concentrations were measured by Cytokine Bead Array. Cytokines that had measurable concentrations are shown. (b) Cell proliferation of the cultures was measured by ³H-thymidine incorporation.

FIG. 4 shows that CD45RA and CD127 identify subpopulations in CD4+ cells cocultured with iDC. CD4⁺CD25⁺ (right panels) or CD4⁺CD25⁻ cells (left panels) were purified from human blood, and cocultured with SB cells for three days. Cells were stained with anti-CD25, CD45RA and CD127.

FIG. 5 shows that CD45RA and CD127 identify subpopulations in freshly isolated CD4⁺CD25⁺ cells. CD4⁺CD25⁺ (right panels) or CD4⁺CD25⁻ cells (left panels) were purified from human blood. Cells were stained with anti-CD25, CD45RA and CD127.

FIG. 6 shows that CD4⁺CD25⁺CD45RA⁻CD127⁻ cells are the only subpopulation from peripheral blood that have Treg cell properties. CD4⁺CD25⁺ cells were purified from human buffy coats by magnetic activated cell sorting (MACS), and stained with primary conjugated anti-CD45RA and anti-CD 127 monoclonal antibodies (mAbs), then sorted by FACS into CD4⁺CD25⁺CD45RA⁺, CD4⁺CD25⁺CD45RA⁻CD127⁺ and CD4⁺CD25⁺CD45RACD127⁻ subpopulations. CD4⁺CD25⁻ cells were separated into CD45RA⁺ and CD45RA⁻ fractions by MACS. Subpopulations were combined with freshly isolated CD4⁺CD25⁻ cells (Tᵣₑₛₚ) at the ratios indicated (Subpopulation to Tᵣₑₛₚ). For stimulators, irradiated allogeneic CD4 depleted PBMCs were used at a ratio of 6:1 stimulators to Tᵣₑₛₚ. Each culture condition was established in triplicate. (a) Concentrations of the indicated cytokines in each culture are shown. Cytokine concentrations were measured by Cytokine Bead Array. Cytokines that had measurable concentrations are shown. (b) Cell proliferation of the cultures was measured by ³H-thymidine incorporation.

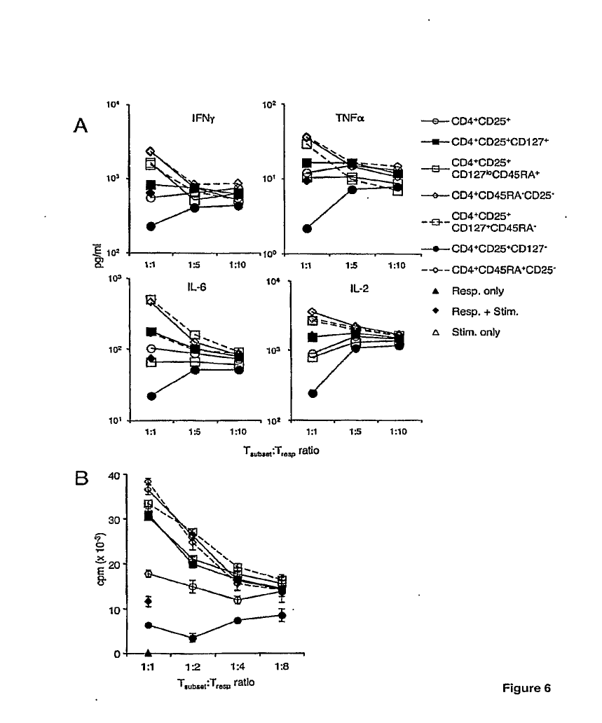

FIG. 7 shows that CD4⁺CD25⁺CD127⁻ cells express higher levels of FoxP3 than CD4⁺CD25⁺CD127⁺ cells. (a) CD4⁺CD25⁺ (right half panels) and CD4⁺CD25⁻ (left half panels) cells were purified from peripheral blood. Samples were stained with anti-CD45RA and anti-CD127 mAbs. Cells were fixed and permeablized, then stained with an anti-FoxP3 mAb. Cells were then analyzed by flow cytometry. Lymphocytes were gated based on forward and side scatter, then further gated based on expression of CD45RA. The percentages of CD45RA⁺ and CD45RA⁻ cells in the CD4⁺CD25⁻ and CD4⁺CD25⁺ fractions are shown in the top histograms. The level of FoxP3 and CD127 expression are shown in the panels. For negative specificity controls, CD4⁻ PBMC were stained with CD127 and either CD19 or CD3. The expression of FoxP3 and CD127 on CD19⁺-gated CD4⁻ PBMCs is shown in panel A. The expression of FoxP3 and CD127 on CD3⁺-gated CD4⁻ PBMCs (presumably cytotoxic T cells) is shown in panel A. For a negative, non-specific control, CD4⁺CD25⁺ cells were stained with the relevant isotype control. (b) CD4⁺CD25⁺ lymphocytes were gated on CD127⁻ cells, then on either CD45RA⁺ (dashed histogram) or CD45RA⁻ (bold histogram) expression, and FoxP3 expression was analyzed. The level of expression of FoxP3 by CD4⁺CD25⁺CD45RA7CD127⁻ (bold histogram) is higher than by CD4⁺CD25⁺CD45RA⁺CD127⁻ (dashed histogram) cells. Isotype control staining by CD4⁺CD25⁺CD45RA7CD127⁻ cells is shown in the light histogram.

### DETAILED DESCRIPTION OF THE INVENTION

The invention provides methods for isolating human regulatory T cell enriched compositions, the resultant compositions and methods of use. In one embodiment, the invention provides a method of suppressing an autoimmune reaction in a subject, the method comprising obtaining a population of regulatory T cell enriched composition from the population of cells; and introducing the population of regulatory T cells into the subject to suppress the autoimmune reaction in the subject.

As described above, Treg cells can be operationally characterized by cell surface markers. These cell surface markers can be recognized by reagents that specifically bind to the cell surface markers. For example, proteins, carbohydrates, or lipids on the surfaces of Treg cells can be immunologically recognized by antibodies specific for the particular protein or carbohydrate (for use of antibodies to markers, see, Harlow, Using Antibodies: A Laboratory Manual (Cold Spring Harbor Press, Cold Spring Harbor, N.Y., 1999); see also, EXAMPLES). The set of markers present on the surfaces of Treg cells and absent from the surfaces of these cells is characteristic for Treg cells. Therefore, Treg cells can be selected by positive and negative selection using cell surface markers. A reagent that binds to a cell surface marker expressed by a Treg cell, a “positive marker”, can be used for the positive selection of Treg cells (i.e., retaining cells that express the cell surface marker). Conversely, negative selection relies on that fact that certain cell surface markers are not expressed by Treg cells. Therefore, a “negative marker” (i.e., a marker not present on the cell surfaces of Treg cells) can be used for the elimination of those cells in the population that are not Treg cells by the removal of cells that bind to the reagent specific for the negative marker.

In one embodiment, discrimination between cells based upon the detected expression of cell surface markers occurs by comparing the expression of a cell surface marker with the mean expression by a control population of cells. For example, the expression of a marker on a Treg cell can be compared to the mean expression of the same marker on other cells derived from the same sample as the Treg cell. Other methods of discriminating among cells by marker expression include gating cells by flow cytometry using a combination of reagents (see, Givan A, Flow Cytometry: First Principles, (Wiley-Liss, New York, 1992); Owens M A & Loken M R., Flow Cytometry: Principles for Clinical Laboratory Practice, (Wiley-Liss, New York, 1995)).

By a “combination of reagents” is meant at least two reagents that bind to cell surface markers either present (positive marker) or not present (negative marker) on the surfaces of Treg cells, or that bind to a combination of positive and negative markers. For example, the use of a combination of antibodies specific for Treg cell surface markers results in isolation and/or enrichment of Treg cells from a variety of samples/tissues.

By selecting for phenotypic characteristics among the cells obtained from the sample, antibodies that recognize species-specific varieties of markers are used to enrich for, and select Treg cells. For example, antibodies that recognize the species-specific varieties of CD4, CD25, CD45RA, CD127 and other markers will be used to enrich for or isolate Treg cells from that species (for example, antibody to a human CD4 for human Treg cells).

“Enriched”, as in an enriched population of cells, can be defined based upon the increased number of cells having a particular marker in a fractionated set of cells as compared with the number of cells having the marker in the unfractionated set of cells. In particular embodiments, the Treg cells are enriched from a population of cells using reagents that bind cell surface markers specific for Tregs and separating these cells using cell sorting assays such as fluorescence-activated cell sorting (FACS), solid-phase magnetic beads, etc, as described below in the EXAMPLES. In some embodiments, combinations of methods to sort the cells can be used, e.g., magnetic selection, followed by FACS. To enhance enrichment, positive selection is combined with negative selection for Treg cell isolation using surface markers such as CD4, CD25, CD45RA and CD127.

It is intended that isolation/enrichment of Treg cells using cell surface markers can be performed in any order. Therefore, a positive selection step may immediately precede a negative selection step, or vice versa. It is also contemplated that isolation/enrichment be performed by grouping the positive selection and negative selection steps. Therefore, isolation/enrichment is done by first performing the positive selection steps of the method, followed by performing the negative selection steps of the method, or vice versa. In one embodiment of the invention, a population of cells is first contacted with reagents that bind CD4 and CD25, followed by reagents that bind CD45RA and CD127. In another embodiment, a population of cells is first contacted with reagents that bind CD4 and CD25, followed by reagents that bind CD127. In another embodiment, a population of cells is contacted with reagents that bind CD45RA and CD127, followed by reagents that bind CD4 and CD25. In yet another embodiment, a population of cells is contacted with reagents that bind CD128, followed by reagents that bind CD4 and CD25. In a further embodiment, a population of cells is sequentially contacted with a first, second, third and fourth reagent that binds CD4, CD25, CD45RA and CD127, respectively.

It is also possible to enrich for CD4⁺ cells by depleting non-CD4⁺ immune cells. Such cell types include, but are not limited to, cytotoxic T cells, γ/δ T cells, B cells, natural killer cells, dendritic cells, monocytes, granulocytes and erythroid cells. Non-CD4⁺ immune cell markers include, but are not limited to, CD8, CD14, CD16, CD19, CD36, CD45RA, CD56, CD123, TCR γ/δ and glycophorin A. In one embodiment of the invention, a population of cells is first contacted with a first reagent or group of reagents that bind one or more of CD8, CD14, CD16, CD19, CD36, CD56, CD123, TCR γ/δ and glycophorin A, followed by reagents that respectively bind CD25, CD45RA and CD127. In another embodiment, a population of cells is contacted with a first reagent or group of reagents that binds the non-CD4⁺ immune cell markers CD8, CD14, CD16, CD19, CD36, CD56, CD123, TCR δ/δ and glycophorin A; and a second, third and fourth reagent that respectively bind CD25, CD45RO and CD127.

CD45 is a gene specifically expressed in hematopoietic cells and has been shown to be an essential regulator of T- and B-cell antigen receptor signaling. Four isoforms of CD45 have been reported, of which CD45RA and CD45RO are two. CD45RO is expressed in subsets of T-cells and B-cells, monocytes, and macrophages. CD45RA is expressed in B-cells, naive T-cells, and monocytes. In one embodiment, Treg cells are isolated/enriched by selecting cells that do not express CD45RA. In another embodiment, Treg cells are isolated/enriched by selecting cells that express CD45RA. In yet another embodiment, Treg cells are isolated/enriched by selecting cells that express CD45RO.

As further described below, depletion of non-regulatory T cells can also be used to enrich for Treg cells. For instance, Treg cell enrichment can be performed by selectively depleting cells that are positive for non-regulatory T cell markers. In one embodiment of the invention, regulatory T cells are enriched by the removal of cells that are positive for one or more of CD8, CD14, CD16, CD19, CD36, CD56, CD123, TCR γ/δ glycophorin A and CD45RA. In another embodiment, cells are further depleted by removal of CD127⁺ cells.

A Treg cell enriched composition is one in which the percentage of Treg cells is higher than the percentage of Treg cells in the originally obtained population of cells. Although possible, an enriched population of Treg cells need not contain a homogenous population of Treg cells. In particular embodiments, at least about 50%, about 55%, about 60%, about 65%, about 70%, about 75%, about 80%, about 85%, about 90%, about 95%, about 98%, or about 99% of said cells of the composition are regulatory T cells.

Any cellular source that contains T cells can be used to isolate/enrich Treg cells. Useful sources include, but are not limited to, peripheral blood, synovial fluid, spleen, thymus, lymph nodes, bone marrow, Peyer's patches, and tonsils.

Enrichment methods are variable based on the level of enrichment associated with each step of the enrichment process. The level of enrichment and percent purity of the Treg cells will depend on many factors including, but not limited to, the donor, the cell/tissue source and the disease state of the donor. In particular embodiments, the Treg cells are enriched at least about 2-fold, about 5-fold, about 10-fold, about 15-fold, about 20-fold, about 25-fold, about 30-fold, about 35-fold, about 40-fold, about 50-fold, about 55-fold, about 60-fold, about 65-fold, about 70-fold, about 75-fold, about 80-fold, about 85-fold, about 90-fold, about 95-fold, about 100-fold, about 105-fold, about 110-fold, about 115-fold, about 120-fold, about 130-fold, about 140-fold, about 150-fold, or about 200-fold.

Enrichment methods of PBMCs using negative selection of CD4⁺ cells alone can enrich regulatory T cells approximately 6-fold. Enrichment methods using CD4 and CD25 can enrich for Treg cells approximately 60-fold. Enrichment using additional markers (such as CD45RA and CD127) can enrich approximately 120-fold or more and can be used to isolate regulatory T cells. Table 1 is a representative purification scheme showing the degree of homogeneity obtainable using whole blood as the starting material. As mentioned above, the degree of homogeneity and fold enrichment vary depending on the starting material and Table 1 is provided for illustrative purposes and is not intended to be limiting.

| Purification step | Cell Population Isolated | Estimated % T_{regs} |
|---|---|---|
| | Peripheral Blood white cells | 0.12-0.3% |
| Ficoll | PBMCs | 0.6-1.5% |
| MACS¹ | CD4⁺ | 3-5% |
| MACS² | CD4⁺CD25⁺ | 30-50% |
| FACS | CD4⁺CD25⁺CD45RA⁻CD127⁻ | 75-90% |

¹CD4⁺ negative selection kit and
²CD25⁺ positive selection kit (Miltenyi Biotec Inc., Auburn, CA)

“Isolated” refers to a cell that is removed from its natural environment (such as in peripheral blood) and that is isolated or separated, and is at least about 75% free, and most preferably about 90% free, from other cells with which it is naturally present, but which lack the cell surface markers based on which the cells were isolated.

Procedures for separation include, but are not limited to magnetic separation using antibody-coated magnetic beads (Schwartz, et al, U.S. Pat. No. 5,759,793) and affinity chromatography or “panning” using antibody attached to a solid matrix (e.g. a plate). Further techniques providing accurate separation include fluorescence-activated cell sorters (FACS), which can have varying degrees of sophistication, such as having multiple color channels, low angle and obtuse light scattering detecting channels, or impedance channels. Dead cells can be eliminated by selection with dyes associated with dead cells e.g., (propidium iodide, LDS). Red blood cells can be removed by, for example, elutriation, hemolysis, or Ficoll-Paque gradients. Any technique can be employed that is not unduly detrimental to the viability of the selected cells.

Conveniently, antibodies can be conjugated with labels for a number of different purposes: e.g., magnetic beads to allow for ease of separation of Treg cells; biotin, which binds with high affinity to avidin or streptavidin; fluorochromes, which can be used with a fluorescence activated cell sorter; haptens; and the like. Multi-color analyses can be employed with FACS or in a combination of immunomagnetic separation and flow cytometry. Multi-color analysis is of interest for the separation of cells based on multiple surface antigens: e.g., non-CD4⁺ immune cell markers (CD8, CD14, CD16, CD19, CD36, CD56, CD123, TCR γ/δ and glycophorin A), CD4⁺, CD25⁺, CD45RA⁻ and CD127⁻. Fluorochromes which find use in a multi-color analysis include, but are not limited to, phycobiliproteins, e.g. phycoerythrin and allophycocyanins; fluorescein, and Texas red.

Magnetic separation is a process used to selectively retain magnetic materials within a vessel, such as a centrifuge tube or column, in a magnetic field gradient. Treg cells can be magnetically labeled by binding magnetic particles to the surface of the cells through specific interactions, including immuno-affinity interactions. The suspension, containing the Treg cells within a suitable vessel, is then exposed to magnetic field gradients of sufficient strength to separate the Treg cells from other cells in the suspension. The vessel can then be washed with a suitable fluid to remove the unlabeled cells, resulting in a purified suspension of Treg cells.

The majority of magnetic labeling systems use superparamagnetic particles with monoclonal antibodies or streptavidin covalently bound to their surface. In cell separation applications, these particles can be used for either positive selection, where the cells of interest are magnetically labeled and retained, or negative selection where the majority of undesired cells are magnetically labeled and retained. The diameter of the particle used varies widely from about 50-100 nm for MACS particles (Miltenyi Biotec) and StemSep™ colloid (StemCell Technologies), through 150-450 nm for EasySep® (StemCell Technologies) and Imag particles (BD Biosciences), up to 4.2 μm for Dynabeads (Dynal Biotech). The type of particle used is influenced by the magnet technology employed to separate the labeled cells.

There are two important classes of magnetic separation technologies, both of which, for convenience and for practical reasons, use permanent magnets as opposed to electromagnets. The first class is column-based high-gradient-magnetic-field separation technology that uses small, weakly magnetic particles to label the targets of interest, and separates these targets in a column filled with a magnetizable matrix. Very high gradients are generated close to the surface of the matrix elements when a magnetic field is applied to the column. The high gradients are necessary to separate targets labeled with these relatively weakly magnetic particles. The second class is tube-based technology that uses more strongly magnetic particles to label the targets of interest. These targets of interest are then separated within a centrifuge-type tube by magnetic field gradients generated by a magnet outside the tube. This method has the advantage that it does not rely on a magnetizable matrix to generate the gradients; and, therefore does not require an expensive disposable column or a reusable column with an inconvenient cleaning and decontamination procedure.

Once placed within the magnet, targeted cells migrate toward the region or regions of highest magnetic field strength and are retained within the magnetic field while the unlabeled cells are drawn off. The targeted cells can then be collected and used after removal from the magnetic field. In the event that negative selection is required, the unlabeled cells are retained and can be utilized for a variety of applications.

FACS permits the separation of sub-populations of cells on the basis of their light scatter properties as they pass through a laser beam. The forward light scatter (FALS) is related to cell size, and the right angle light scatter, also known as side scatter characteristic (SSC) is related to cell density, cellular content and nucleo-cytplasmic ratio, i.e. cell complexity. Since cells can be labeled with fluorescent-conjugated antibodies, they can further be characterized by antibody (fluorescence) intensity.

In particular embodiments, the Treg cells of the present invention, are isolated using immnuomagnetic chromatography. For example, an anti-CD4 antibody is attached to magnetic beads. These antibody-labeled magnetic beads are used as the basis for the affinity purification. The antibody-labeled fraction of T cells are applied to the magnetic affinty column. The non-adherent cells are discarded and the adherent cells are eluted from the magnetic column by removal of the magnetic field. In another embodiment, the cells are first labeled with an antibody (e.g., anti-CD4) and then labeled with a secondary antibody carrying a magnetic bead or sphere.

In another embodiment, a secondary antibody immunoreactive with a Treg cell can be used to enrich the population of Treg cells. The use of a secondary antibody is generally known in the art. Typically, secondary antibodies are antibodies immunoreactive with the constant regions of the first antibody. Preferred secondary antibodies include anti-rabbit, anti-mouse, anti-rat, anti-goat, and anti-horse immunoglobulins and are available commercially. Commercially available kits provide secondary antibodies conjugated to labeling agents such as, but limited to, magnetic particles and fluorochromes.

In some embodiments, the population of cells is obtained from the subject, obtained from a donor distinct from the subject, and/or harvested from peripheral blood. The population of cells obtained comprises regulatory T cells, and may be derived from any source in which Treg cells exist, such as peripheral blood, the spleen, thymus, lymph nodes, bone marrow, Peyer's patches and tonsils.

In another embodiment, the invention is directed to the treatment of an autoimmune disease by administration of regulatory T cells. As has been shown previously, mice deficient for Treg cells develop and succumb to multi-organ autoimmune disease (Asano et al., 1996; Ramsdell et al., 2003). Furthermore, in U.S. Pat. Appl. Pub. No. 2005/0186207 A1, incorporated herein by reference, Treg cells are predicted to suppress autoimmunity. Therefore, isolated/enriched populations of regulatory T cells of the invention can be used to suppress autoimmune disease.

In general, autoimmune responses occur when the immune system of a subject recognizes self-antigens as foreign, leading to the production of self-reactive effector immune cells. Self-reactive effector cells include cells from a variety of lineages, including, but not limited to, cytotoxic T cells, helper T cells, and B cells. While the precise mechanisms differ, the presence of autoreactive effector cells in a host suffering from an autoimmune disease leads to the destruction of tissues and cells of the host, resulting in pathologic symptoms. Numerous assays for determining the presence of such cells in a host, and therefore the presence of an autoimmune disease, such as an antigen specific autoimmune disease in a host, are known to those of skill in the art and readily employed in the subject methods. Assays of interest include, but are not limited to, those described in: Autoimmunity 36:361-6 (2003); J Pediatr Hematol OncoL 25 Suppl 1:S57-61 (2003); Proteomics 3:2077-84 (2003); Autoimmun. Rev. 2:43-9 (2003).

The population of cells may be obtained from the subject into which the Treg-enriched composition is subsequently introduced. The subject can be one in which suppression of an autoimmune reaction is desired. In one embodiment, the subject is a human afflicted with an autoimmune disease or disorder, such as any of the diseases/disorders including, but not limited to: lupus erythematosus, pemphigus vulgaris, thyreoiditis, thrombocytopenic purpura, Graves disease, diabetes mellitus, myasthenia gravis, Addison's disease, rheumatoid arthritis, multiple sclerosis, psoriasis, uveitis, and autoimmune hemolytic anemia. The cells of the invention can also be used to prevent or treat transplantation reactions such as graft versus host disease (GVHD) and graft rejections.

Introduction of Treg cells into patients is performed using methods well known in the art such as adoptive cell transfer. Briefly, a mixed population of cells is extracted from a target donor. Depending on the application, the cells may be extracted during a period of remission, or during active disease. Typically this is done by withdrawing whole blood and harvesting granulocytes by leukapheresis (leukopheresis). For example, large volume leukapherisis (LVL) has been shown to maximize blood leukocyte yield. The harvested lymphocytes may be separated using the cell separation techniques based on Treg-specific cell markers such as those described herein, and then transfused to a patient, typically the cell donor (except in GVHD where the donor and recipient are different), for adoptive immune suppression. Approximately 10⁹ to 10¹¹ Tregs cells are transfused into the patient.

By treatment is meant that at least an amelioration of the symptoms associated with the autoimmune response in the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

### Regulatory T Cells

The surface phenotype of human peripheral blood Treg cells is identified herein as CD4⁺CD25⁺CD45RA⁻CD127⁻. The data shows that peripheral blood CD4⁺CD25⁺ cells are heterogenous, and by short-term coculture of T cell and immature dendritic cell (iDC), at least two populations can be observed. Furthermore, by using markers that distinguish naive cells, this population can be phenotypically dissected and at least three subpopulations are found. Functionally, the suppressive activity resides in only one of these three populations.

The degree of heterogeneity of the CD4⁺CD25⁺ population is controversial. In mice, it appears that the majority of CD4⁺CD25⁺ cells found in spleen and lymph nodes are Treg cells, as determined by FoxP3 expression. However, in humans, the answer is less clear. CD4⁺CD25^{bright} population is enriched in cells with regulatory activity, but given that there is no distinct demarcation for “bright” versus “medium”, markers that could clearly distinguish the Treg population from other non-Treg CD4⁺CD25⁺ cells were sought to be identified.

Modified, short-term coculture of CD4⁺CD25⁺ cells with iDC demonstrates that the CD4⁺CD25⁺ population is heterogeneous. After repetitively stimulating purified CD4⁺ cells with iDC for two weeks, a population of CD4⁺CD25⁺ with regulatory activity arose. One possible interpretation of this data is that iDC provided a preferential signal to the Treg cells, and selected the Treg cells to survive. By first enriching for CD4⁺CD25⁺ cells prior to the coculture, iDC were shown to provide signals that enable a subset of CD4⁺CD25⁺ cells to upregulate CD25 expression, whereas another subset downregulates CD25 expression. From this short-term coculture, it appears that the CD25⁺ population is comprised of at least two subpopulations that differ by their ability to upregulate CD25 in the presence of iDC.

By isolating these cells and testing their ability to suppress T cell activation, the subpopulation that upregulates surface CD25 expression are shown to be Treg cells. This observation, therefore is consistent with the observations made in that stimulating CD4⁺PBMC with iDC can result in a population that expresses CD25 and has Treg cell function. The claimed invention builds upon these observations and suggests that iDCs provide a signal to Treg cells present in the population, and selectively cause them to sustain or upregulate CD25 expression.

Ligation of CD28/CD152 on CD4⁺CD25⁺ cells by CD80/CD86 on iDCs may be involved in the ability of iDC to provide selective signals to Treg cells (Saloman, B. et al., Immunity 12:431-440 (2000); Tang, Q. et al., J. Immunol. 171:3348-3352 (2003)). In agreement with this, SB B-lymphoblastoid cell line were found to mimic the ability of iDC to upregulate CD25 expression on a subset of CD4⁺CD25⁺, and soluble CD152/Ig can inhibit CD25 upregulation, presumably by blocking CD80/CD86 interaction with CD28/CD152. This result is consistent with previous reports arguing for a role of CD28 stimulation on Treg cells in their development. However, in the in vitro system of this invention, it could not be distinguished whether signaling via CD28, CD152 or both, causes CD25 upregulation.

By analyzing the subsets from the coculture for CD45RA expression, CD4⁺CD25⁺ population can be further segregated, further arguing that this is a heterogeneous population. In some donors, the majority of CD45RA cells cosegregate with the CD25⁺ population, but from other donors, CD45RA⁺ cells are found in both CD25⁺ and CD25⁻ populations. It is not known what causes the variability among donors. CD45RA is believed to be a marker for naive cells, and it is presumed that the CD4⁺CD25⁺CD45RA⁺ cells found in PBMC are activated naive T cells.

Of the two cocultured subpopulations, the cells which upregulated CD25⁺ expression were enriched for Treg cells, and this supported by the fact that these cells retained regulatory activity. The identity of the subpopulation that downregulated CD25 expression was also determined. Because CD4⁺CD25⁺ predominantly express CD45RO, it was hypothesized that the second cocultured subpopulation could be memory T cells. A variety of markers differentially expressed by memory cells was analyzed, including CCR7, CD62L, CD69, but the marker that gave the most startling pattern of expression was CD127. In the cocultured cells an inverse correlation of CD127 expression compared to CD25 expression was observed.

CD25 and CD127 are a-chains of heterotrimeric receptors. They complex with a β-chain and the common γ-chain signaling subunit, forming receptors for IL-2 or IL-7 respectively. IL-7 has historically been associated with lymphopoiesis; however, recently, it has been implicated in the development of memory cells (Kondrack, R. M. et al., J. Exp. Med. 198:1797-1806 (2003); Li, J. et al., J. Exp. Med. 198:1807-1815 (2003)), and in the homeostatic proliferation of peripheral CD4⁺ and CD8⁺ T cells (Schluns, K. S. et al., Nat. Immunol. 1:426-432 (2000); Tan, J. T. et al., J. Exp. Med. 195:1523-1532 (2002)). IL-2 has historically been associated with T cell activation, and recently, its role in maintaining the peripheral numbers of Treg cells has been recognized (Fontenot, J. D. et al., Nat. Immunol. 6:1142-1151 (2005)). CD127 also serves as the thymic stromal lymphopoeitin (TSLP) receptor, however, the physiologic role of TSLP in human mature T cell function remains to be elucidated. The CD4⁺CD25⁺ population will also be analyzed for the expression of other α-chains that pair with the common γ-chain, such as the IL-15 receptor, when these reagents become available.

Lower CD127 expression by the CD25^{high} CD4⁺ cells is also observed on freshly isolated CD4⁺CD25⁺ PBMC. Combined with CD45RA expression, the CD4⁺CD25⁺ PBMC population can be segregated into three subpopulations. The CD4⁺CD25⁺CD45RA⁻CD127⁻ subpopulation was the only one capable of suppressing T cell proliferation and cytokine production in an allostimulation assay. Furthermore, when analyzed for FoxP3 expression, the majority of FoxP3⁺ cells in the CD25 fraction were CD127⁻. Therefore, because these cells functionally and phenotypically resemble Treg cells, the surface phenotype of human Treg cells is CD4⁺CD25⁺CD45RA⁻ CD127⁻.

FoxP3 was also detected in CD4⁺CD25⁺CD45RA⁺CD127⁻ cells. These cells display characteristics that distinguish them from the CD4⁺CD25 CD45RACD127⁻ population. FoxP3 was also expressed at a lower level in the CD4⁺CD25⁺CD45RA⁺CD127⁻ subpopulation. Also, the frequency of this population was the most varied between donors. On average, this population represents 13.4% of the CD25⁺ enriched cells (SD=7, n=10), however, depending on the donor, frequencies ranging from 6% to 28% were observed. Finally, the CD4⁺CD25⁺CD45RA⁺ subpopulation was not observed to suppress effector T cell proliferation in suppression assays, and the CD127^{lo} cells generally comprised over half of the population. If the CD4⁺CD25⁺CD45RA⁺CD127⁻ are Treg cells, the entire CD4⁺CD25⁺CD45RA⁺ subpopulation should exert some suppressive function because even at ratios of 1:2 to 1:5, the CD4⁺CD25⁺CD45RA⁻CD127⁻ cells can suppress T effector cell cytokine production and proliferation. On occasion, the CD4⁺CD25⁺CD45RA⁺ cells were able to suppress cytokine production in suppression assays, however, this result was not consistent.

However, it is possible that this subpopulation of cells are also Treg cells. A subset of CD4⁺CD25⁺CD45RA⁺T cells were recently characterized from cord blood that expressed FoxP3 and functionally suppressed in vitro Tᵣₑₛₚ cell proliferation (Seddiki, N. et al., Blood (2005, in press)). It remains to be seen whether the CD4⁺CD25⁺CD45RA⁺ cells found in adult peripheral blood have the same suppressive properties. Because the FoxP3⁺, CD4⁺CD25⁺CD45RA⁺CD127^{lo} cells are present in such small numbers, it will be technically challenging to demonstrate this.

CD127 downregulation has been described to be a consequence of T cell activation either by immunization with virus in vivo, or in vitro crosslinking of CD3 and CD28, or antigenic stimulation (Li et al., 2003; Xue, H. H. et al. Proc. NatL Acad. Sci. USA 99:13759-13764 (2002)). Also, modulation of CD127 expression is further observed in the presence of IL-2. Interestingly in the mouse, modulation of FoxP3 expression by Treg cells appears to also be directly or indirectly affected by IL-2 signaling. The present invention demonstrates that FoxP3 expressing cells have downregulated CD127 expression. Also, CD4⁺CD25⁺CD45RA⁻CD127 cells express higher levels of FoxP3 than the CD4⁺CD25⁺CD45RA⁺CD127^{lo} cohorts, and only the former subpopulation has potent regulatory activity. These results may reflect that, of the CD4⁺CD25⁺ peripheral blood T cells, Treg cells are chronically stimulated with IL-2. In contrast, the other CD25⁺ populations may represent recently activated T cells because they have not either downregulated CD127 expression, or have a lower level of FoxP3 expression.

It is interesting to note the differences between mouse CD4⁺CD25⁺ T cells and human CD4⁺CD25⁺ T cells. In the mouse, all CD4⁺CD25⁺ cells appear to express FoxP3 (Fontenot et al., 2005). In contrast, this subpopulation comprised about 40-50% of the human CD4⁺CD25⁺ population. The presence of CD25⁺ non-Treg cells in humans may reflect the fact that humans are constantly exposed to environmental pathogens, whereas mice are maintained in a controlled environment.

This combination of extracellular markers is an unambiguous strategy to identify and purify live, human Treg cells from PBMC for use in functional assays. Other markers that are upregulated on Treg cells have been reported, but so far only in the mouse system. In humans, differential expression of PD-1, GITR, sTGF-(3 (LAP), CD103, CD152 and LAG-3 was studied, but none of the markers has been able to subdivide the CD4⁺CD25⁺ population into ones that have suppressive functions. It has been shown that Treg cells in synovial joint infiltrate of rheumatoid arthritis patients expressed CD27 in contrast to activated T cells that were CD27⁻ (Ruprecht et al. 2005). The same difference in CD27 expression on circulating CD4⁺CD25⁺ PBMC was not observed. It is possible that the surface phenotype of activated CD4⁺CD25⁺ cells change as they migrate from the circulation into sites of inflammation.

### EXAMPLES

### CD4⁺CD25⁺ T Cells can be Segregated by Coculture with Allogeneic iDC

It has been shown that longterm coculture and repetitive stimulation of human CD4⁺PBMC with in vitro-generated iDC can support the development of T cells that had regulatory activity (Jonuleit et al. 2000). These iDC primed CD4⁺ T cells have many properties that are consistent with Treg cells. For example, these iDC primed CD4⁺ T cells express CD25 and CD152, they do not proliferate to allogeneic stimulation, they are responsive to high-levels of IL-2, and, importantly, they mediate contact-dependent suppression of responder T (Tᵣₑₛₚ) cells. These results suggest that these in vitro derived, regulatory-like T cells were induced by iDC, and in the presence of iDC, naive T cells can be converted into T cells with regulatory properties (Jonuleit et al. 2000). However, there is an increasing body of evidence that Treg cells represent a distinct lineage of T cells emerging from thymocyte selection (Fontenot et al. 2005). Another interpretation of this data is that iDC could select existing Treg cells from CD4⁺ T cell population, and promote their survival. Interestingly, during the long-term cocultures, a decrease in overall T cell numbers was observed, which is consistent with the possibility that a subpopulation was being selected from the CD4⁺ T cell population (Jonuleit et al. 2000). It was hypothesized that coculture of CD4⁺ cells with iDC could select cells with Treg cell activity.

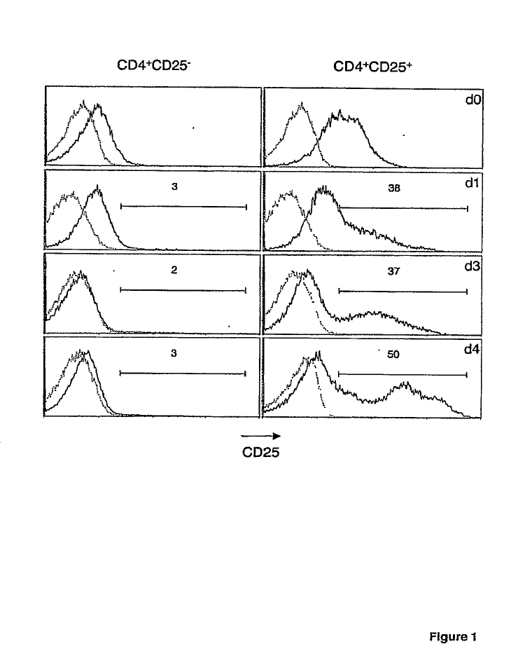

It was reasoned that if iDC could promote Treg cell survival, enriching the CD4⁺ population into CD25⁺ or CD25⁻ prior to iDC priming would allow for tracking the development of Treg cells. Also, if repetitive stimulation of naive CD4⁺ cells by allogeneic iDC was inducing development of a regulatory-like T cell population that upregulated CD25 expression, increased CD25 expression in the CD4⁺CD25⁻ enriched population over a short term coculture could be observed. CD4⁺CD25⁺ and CD4⁺CD25⁻ cells were enriched from CD4⁺ T cells by MACS. Either CD4⁺CD25⁺ or CD4⁺CD25⁻ cells with allogeneic iDCs, derived from 7 day culture of CD14⁺PBMCs with IL-4 and GM-CSF, were cocultured. Initially, MACS enriched a population of CD4⁺CD25⁺ cells appearing to have a uniform level of CD25 expression, as determined by flow cytometry (FIG. 1). However, over a three day time course, two major populations emerged from the original CD25⁺ population (FIG. 1). One population retained CD25 expression (referred to as CD4⁺CD25^{+>+}), and the other had decreased CD25 expression (referred to as CD4⁺CD25^{+>-}) (FIG. 1). Control CD4⁺CD25⁻ cells cocultured with iDC over the same time course also resulted in two populations: a minor population that upregulated CD25 (referred to as CD4⁺CD25^{+>+}), and another that did not (referred to as CD4⁺CD25^{->-}). The level of CD25 expression on CD4⁺CD25^{+>-} cells was similar to CD4⁺CD25^{->-} cells. The short-term coculture of CD4⁺CD25⁺ T cells with iDC demonstrated that this population is heterogenous with respect to its ability to express CD25 after a 3 day pulse with iDC and suggested that iDC had differential effects on either subpopulation within the CD4⁺CD25⁺ aggregate population. This also raised the possibility that iDC could provide signals that either caused one subpopulation to retain and upregulate CD25 expression, or conversely caused the other subpopulation to downregulate CD25 expression.

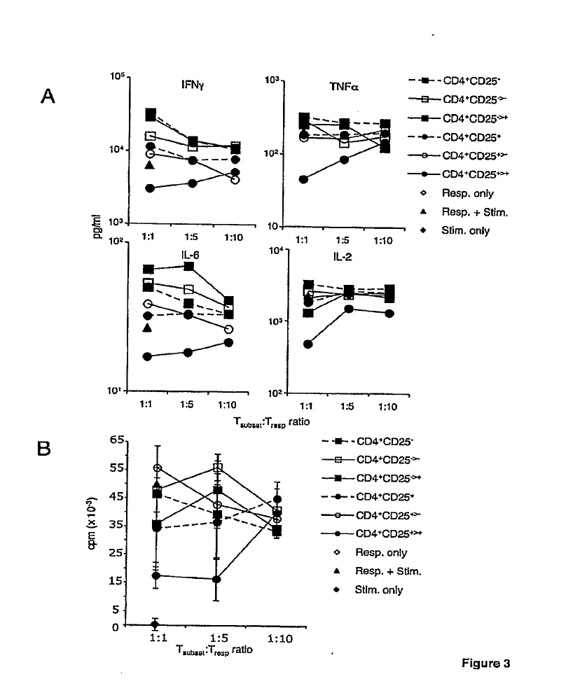

CD4⁺CD25⁺ cells derived from long-term coculture with iDC were observed to have regulatory activity (Jonuleit et al. 2000). Using the short-term iDC pulse, a subpopulation originating from the CD4⁺CD25⁺ PBMC was tested to determine if it was capable of suppressing T cell activation. After priming enriched CD4⁺CD25⁺ T cells for 3 days with iDC, the resulting CD4⁺CD25^{+>+} and CD4⁺CD25^{+>-} subpopulations were purified by FACS, then assayed for suppressive function in a modified allostimulation assay. In this assay, fresh, autologous CD4⁺CD25⁻ were isolated and used as Tᵣₑₛₚ cells in cultures with irradiated, allogeneic, CD4-depleted PBMC. Suppression was measured by the ability of a subpopulation to inhibit Tᵣₑₛₚ cell activation, as indicated by proliferation or cytokine production. At a ratio of 1:1 iDC-primed cells to Tᵣₑₛₚ cells, only CD4⁺CD25^{+>+} cells could mediate suppression of Tᵣₑₛₚ cytokine production and cell proliferation in a dose-dependent manner (FIGS. 3A, B). The internal control (CD4⁺CD25^{+>-}) and negative control cell populations (CD4⁺CD25^{->-}, CD4⁺CD25^{->+}) did not exert any suppressive function on the Tᵣₑₛₚ cells. Freshly isolated CD4⁺CD25⁺ cells were not as potent in suppressing Tᵣₑₛₚ cells as the CD4⁺CD25^{+>+} subpopulation. Therefore, the CD4⁺CD25⁺ cells that continue to express CD25 after short-term iDC coculture are enriched for Treg cells. These experiments revealed the heterogeneity of the CD4⁺CD25⁺ T cell population in terms of the response to iDC priming that was observed phenotypically and functionally.

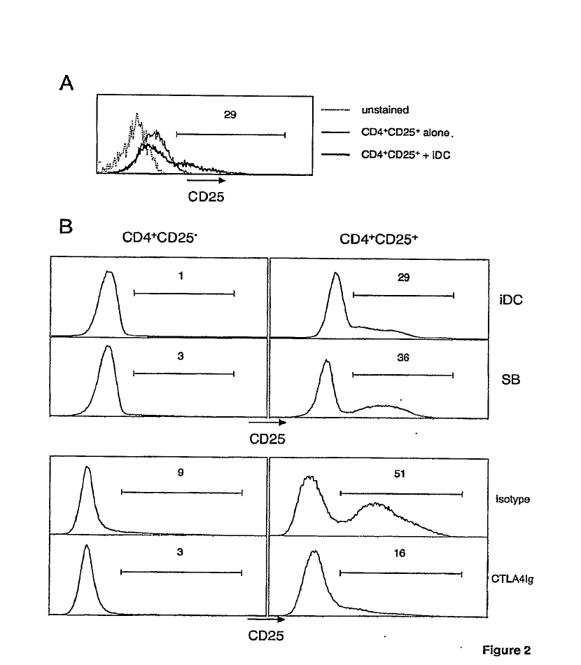

Next, whether the segregration of CD4⁺CD25^{+>+} and CD4⁺CD25^{+>-} cell populations was dependent on coculture with iDC, i.e., if a “tolerogenic” signal via ligands or cytokines was necessary for supporting CD4⁺CD25^{+>+} cell survival was analyzed. One possibility was that the signals provided by the iDC were causing one subpopulation to retain or upregulate CD25 expression. On the other hand, iDC could provide signals that caused the other subpopulation to downregulate CD25 expression. First, CD4⁺CD25⁺ cells were cultured in media alone for 3 days. As shown in FIG. 2A, the level of CD25 expression was comparable to the level expressed by CD4⁺CD25^{+>-} cells. This experiment suggested that iDC provided signals that support CD4⁺CD25⁺ Treg cell development from the original heterogeneous CD4⁺CD25⁺ PBMC population. In addition, the CD4⁺CD25^{+>-} subpopulation do not appear to be responsive to the iDC signals, and hence down-regulate CD25 expression.

Engagement of either CD28 and/or CD152 by CD80 or CD86 is necessary for normal peripheral Treg cell numbers (Saloman et al. 2000; Tang et al. 2003). Whether upregulation of CD25 expression by the CD4⁺CD25^{+>+} population was dependent on cells expressing CD80 or CD86 (CD80/86) was also investigated. First, CD4⁺CD25⁺ cells were cocultured with the SB human lymphoblastoid cell line. This cell line expresses high levels of both CD80 and CD86, relative to resting B cells (Reff, M. E. et al. Blood 83:435-445 (1994)). As shown in FIG. 3B, the SB cell line was capable of segregating CD4⁺CD25⁺ PBMC into CD4⁺CD25^{+>+} and CD4⁺CD25^{+>-} cell populations after 3 days. Finally, the short-term coculture of CD4⁺CD25⁺PBMC with SB lymphoma cell or iDC, in the absence or presence of soluble chimeric CD152-IgFc (CTLA4-Ig) was repeated, and it was shown that this molecule can prevent the segregation of the CD4⁺CD25^{+>+} and CD4⁺CD25^{+>-} cell populations (FIGS. 3C, 3D). Taken together, these experiments suggested that the ability of iDC to sustain the CD4⁺CD25^{+>+} subpopulation, and allow them to retain or upregulate CD25 express, was mediated in part by their interaction with CD80 or CD86 on antigen presenting cells (APC).

### Surface Phenotype of CD4⁺CD25^{+>-} and CD4⁺CD25^{+>+} Segregated Populations

Human CD4⁺CD25^{+>+} subpopulation were further characterized by staining these cells with markers that have been characterized to be upregulated by mouse Treg cells, such as surface CD152, PD-1, GITR, CD62L, CD103 (Gavin, et al., Nat. Immunol. 3:33-41 (2002)). Variable levels of expression were observed, but none of these markers distinguished the CD4⁺CD25^{+>+} cells from the internal control cells, CD4⁺CD25^{+>-}.

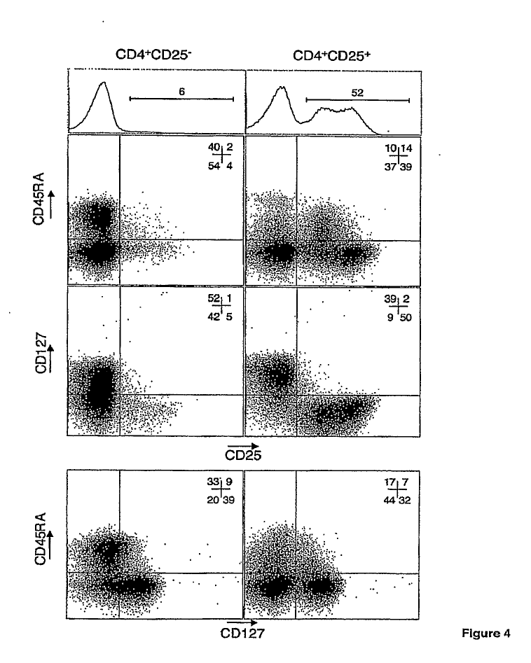

If the CD4⁺CD25⁺ population was heterogenous, and if bona fide Treg cells within this population express unique surface molecules, then the corollary to this hypothesis is that CD4⁺CD25⁺ non-Treg cells would also express unique surface molecules that would differentiate them from Treg cells. One candidate marker is CD45RA. Like others have observed, CD4⁺CD25⁺ PBMCs are predominantly comprised of CD45RA (CD45RO⁺) cells (Baecher-Allan et al. 2001; Jonuleit et al. 2001; Taams et al. 2002). Depending on the donor, it has been observed that approximately 70-80% are CD45RA, with 20-30% CD45RA⁺ cells within this CD4⁺CD25⁺ enriched cells. It is possible that CD4⁺CD25⁺CD45RA⁺ cells represent activated naive T cells, since CD25⁺ is upregulated upon activation (Akbar, A. N. et al., Eur. J. Immunol. 21:2517-2522 (1991)). The subpopulation of CD45RA⁺ cells that would cosegregate after 3 day coculture with iDC was next identified. CD4⁺CD25⁺CD45RA⁺ cells in the CD4⁺CD25⁺ PBMC population have also been shown to lack suppressive function. Therefore, it was predicted that the CD45RA⁺ cells would cosegregate with CD4⁺CD25^{+>-} cells. Interestingly, results varied between donors. From some donors, the majority of CD45RA⁺ cells cosegregated with CD4⁺CD25^{+>-}, and in others, both subpopulations had CD45RA⁺ cells (FIG. 4). While it was not clear what factors accounted for the variability, the CD4⁺CD25^{+>+} cell population which was enriched for Treg cells, was also composed of CD45RA⁺ cells.

CD4⁺CD25⁺ cells are predominantly CD45RA and human memory T cells are also CD45RA⁻; therefore, it was possible that the CD4⁺CD25^{+>-} are memory cells. The CD45RA7 (CD45RO⁺) population can be further subdivided based on CCR7 expression (Sallusto, F. et al., Nature 401:708-712 (1999)). However, in the short-term iDC cocultures, staining the iDC-pulsed CD4⁺CD25⁺ cells was inconclusive because CCR7 appeared to be downregulated after 3 day coculture.

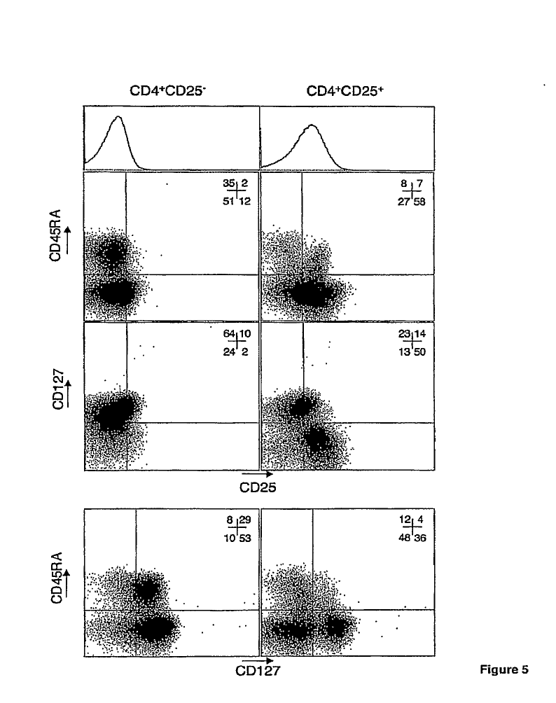

Recent evidence has implicated a critical role for IL-7 in the development and maintenance of memory T cells (Kondrack, R. M. et al., J. Exp. Med. 198:1797-1806 (2003)). Therefore, studies were undertaken to investigate whether the CD4⁺CD25⁺CD45RA cells would differentially express the IL-7Rα (CD127). An inverse correlation of CD127 expression with CD25 expression on CD4⁺CD25⁺ cells cocultured with iDC for 3 days was observed (FIG. 5). Interestingly, in 3 day cocultures of CD4⁺CD25⁻ cells with iDC, almost half of the population was CD127¹⁰ and these CD127^{lo} cells were predominantly CD45RA⁺. Because naive CD4⁺ cells, i.e., CD4⁺CD25⁻ CD45RA⁺ cells, express CD127, coculture with iDC resulted in CD45RA⁺ cells downregulating CD127 expression. Thus, whether low CD127 expression by CD4⁺CD25^{+>+} cocultured cells was a result of CD127 downregulation as a consequence of the short-term iDC pulse, or whether a fraction of CD4⁺CD25⁺ cells in PBMC would exhibit lower CD127 expression levels was analyzed. To test this possibility, freshly isolated CD4⁺CD25⁺ PBMC were stained, and this population, while uniform in CD25 expression, could be separated into two distinct populations, CD127⁺ and CD127^{lo}. In contrast, the majority of CD4⁺CD25⁻ cells were CD127⁺.

CD4⁺CD25⁺CD45RA⁻CD127− T Cells from Peripheral Blood Functionally and Phenotypically Resemble Regulatory T Cells

Thus, based on CD25 staining, the CD25⁺ population appeared to be homogenous. However, using CD45RA and CD127, CD4⁺CD25⁺, PBMC were further separated into 3 distinct populations: CD4⁺CD25⁺CD45RA⁺CD127^{+/lo}, CD4⁺CD25⁺CD45RA⁻CD127⁺ and CD4⁺CD25⁺CD45RA⁻CD127⁻. Coculture data suggest Treg cells reside in the CD127⁻ fraction because after 3 days of priming with iDC, the CD25⁺ cells with regulatory activity were CD127⁻. Reports from independent groups demonstrate that regulatory activity of CD4⁺CD25⁺ T cells resides in the CD4⁺CD25⁺CD45RA cell fraction. Therefore, the CD4⁺CD25⁺CD45RA CD127⁻ subpopulation was a potential candidate for Treg cells. CD4⁺CD25⁺ cells from CD4⁺PBMC were enriched by magnetic bead separation. The CD4⁺CD25⁺ cells were then sorted by FACS into CD4⁺CD25⁺CD45RA⁻CD127⁻, CD4⁺CD25⁺CD45RA⁺CD127^{lo/+} and CD4⁺CD25⁺CD45RA⁻CD127⁺ subpopulations and whether any of the populations had regulatory function was tested. Only CD4⁺CD25⁺CD45RA⁻ CD127⁻ cells were able to consistently suppress proliferation or cytokine production in a dose dependent manner by Tᵣₑₛₚ cells in an allostimulation assay. In contrast, none of the other CD4⁺ subpopulations tested could suppress proliferation or cytokine production below the baseline of Tᵣₑₛₚ cells with stimulators alone. Only the aggregate CD4⁺CD25⁺ cells appeared to have some suppressive function, but they were not as potent as the CD4⁺CD25⁺CD45RA⁻CD127⁻ cells isolated from this fraction. These experiments demonstrate that the CD4⁺CD25⁺ population is comprised of at least three subpopulations that could be segregated based on CD127 and CD45RA expression. Only one of these three subpopulations, namely the CD4⁺CD25⁺CD45RACD127⁻ cells had Treg cell function.

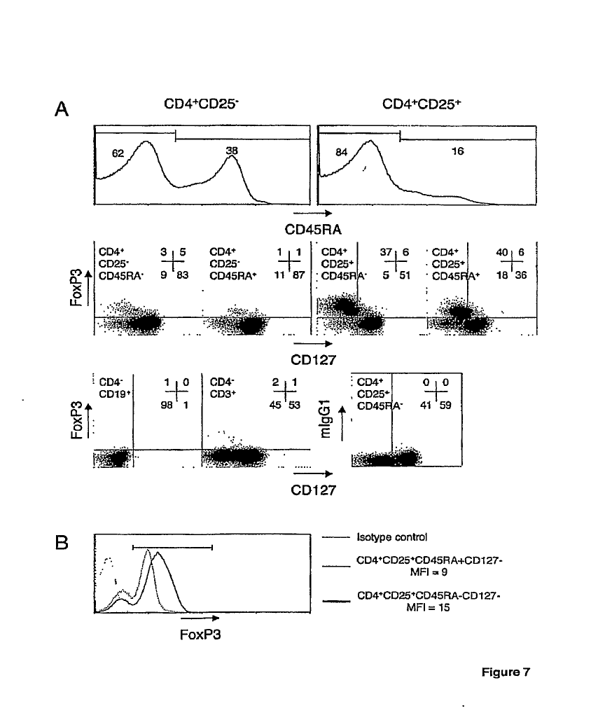

Multiple lines of genetic evidence strongly support the role of the FoxP3 forkhead family transcription factor in the development of the Treg cell population (Fontenot et al. 2005). If CD4⁺CD25⁺CD45RA⁻CD127⁻ PBMCs are Treg cells, then the expression of FoxP3 should be increased in this fraction. The subpopulations of the enriched CD4⁺CD25⁺ PBMC were analyzed for FoxP3 expression by flow cytometry. As shown in FIG. 7, FoxP3 was predominantly expressed by CD4⁺CD25⁺CD127⁻ cells in the CD45RA subpopulation, as determined by intracellular staining. Interestingly, FoxP3 expression was detectable in 40% of CD4⁺CD25⁺CD45RA⁺ cells, despite the fact that CD4⁺CD25⁺CD45RA⁺ cells were not able to suppress cytokine production and cell proliferation by effector T cells. There also appeared to be three major populations within the CD4⁺CD25⁺CD45RA⁺ fraction, based on CD127 and FoxP3 expression. FoxP3 expression was not observed in CD127⁺ and CD127⁻ cells. Similar to the CD4⁺CD25⁺CD45RA⁻ cohorts, FoxP3 expression was observed in CD127^{lo} cells. However, CD4⁺CD25⁺CD45RA⁺CD127^{lo} cells expressed a lower level of FoxP3 relative to CD4⁺CD25⁺CD45RA⁻CD127^{lo} cells, as determined by mean fluorescent intensity (FIG. 7B). Taken together, FoxP3 expression is correlated with CD25⁺ expression and inversely correlated with CD127 expression.

### Methods

Flow Cytometry. Cells were stained using standard procedures. Briefly, cells were suspended at a concentration of 1×10⁷ cells/ml in PBS+3% FBS for analysis or in Sort Buffer (PBS, 25 mM HEPES, 1 mM EDTA, 0.1% BSA) for sorting. The amount of antibody added was in accordance to manufacturer's suggested volume, or was determined by titration. Cells were analyzed by flow cytometry using a FACScalibur (Becton Dickson hnmunocytometry Systems, San Jose, Calif.) and operated under standard procedures. To enrich subpopulations of CD4⁺ cells, magnetically separated cells were stained with anti-CD127⁻ PE and anti-CD45RA-APC, then sorted on a MoFlo (DAKOCytomation, Fort Collins, Colo.) using standard procedures. Antibodies used were CD25-PE, CD25-PECy5, CD25-APC (M-A251), CD45RA-APC (HI100), CD45RAPECy7 (L48), CD127⁻ PE IL-7R-M21) (BD Biosciences Pharrningen, San Diego, Calif.); FoxP3-Alexa488 (206D) (BioLegend, San Diego, Calif.).

Cell Isolation. PBMCs were isolated from blood drawn from donors, or from buffy coats (San Diego Blood Bank, San Diego, Calif.). Approximately 150 ml donor blood was drawn into heparinized blood collection tubes (VWR, West Chester, Pa.), then diluted 1:2 in PBS. Buffy coats were diluted to a final volume of 1 L in PBS. Approximately 3 volumes of diluted sample were layered over 1 volume of Histopaque-1077 (Sigma-Aldrich, St. Louis, Mo.), then centrifuged 1400 rpm, at room temperature for 30 minutes. Cells at the interface were harvested, washed and resuspended in MACS Buffer for further separation.

Cell separation by MACS. Cells were separated by MACS microbeads (Miltenyi Biotec, Auburn, Calif.), following the manufacturer's protocol, using LS columns (Miltenyi Biotec). In order to elute bound cells, the columns were removed from the magnetic field. 3 ml of MACS Buffer was added to the column, and the eluted cells were collected. The following microbead kits were used: CD4⁺ T Cell Isolation Kit II, Human CD25⁺Microbeads, and Human CD45RA Microbeads.

Cell culture. Immature dendritic cells were derived following the protocol described by Joneilut et al. Briefly, CD14⁺PBMCs were resuspended at a concentration of 10⁶ cells/ml in X-VIVO complete media: X-VIVO (Cambrex, Walkersville, Md.), 10% FBS (HyClone, Logan, Utah) 4 mM L-glutamine (Invitrogen, Carlsbad, Calif.), 0.1 M HEPES (Invitrogen), 1×MEM Non-essential amino acids (Sigma, St. Louis, Mo.), 1 mM Sodium Pyruvate (Invitrogen), and antibiotics. For iDC derivation, X-VIVO complete media was supplemented with 150 ng/ml GM-CSF (R&D Systems, Minneapolis, Minn.) and 150 ng/ml IL-4 (R&D Systems). Cells were incubated for 7d at 37° C. Dendritic cells were harvested, washed in PBS and resuspended in X-VIVO complete media. The CD152/IgFc fusion protein was constructed using standard recombinant techniques. The nucleotides encoding residues 1-161 of CD152 were amplified then cloned into the expression vector INPEP4, under control of the CMV promoter and in frame with a modified Fc portion of human IgGl. Cys in the hinge region (corresponding to residues 250, 256 and 259 of the mature IgGl protein) were mutated to Ser to prevent dimerization.

CHO cells were transfected and cell lines stably expressing the protein were selected. CD152/IgFc fusion protein was purified using Protein-A Sepharose columns. Purified CD152/IgFc fusion protein or in supernatant was detected by SDSPAGE, ELISA, and functional assays (T. Snipas and T. Yun, unpublished observations).

Coculture. CD4⁺ T cell subsets were combined with DCs at a ratio of 5:1 T cells-to-DC, or with SB lymphoblastoid cells at a ratio of 10:1 T cells-to-SB cells. The cells were resuspended in X-VIVO complete media at a concentration of 5×10⁶ cells/ml, and cultured for 3 days at 37° C.

Cytometric Bead Array. After approximately 72 hours of culture, 1/3 of the supernatant from each well was sampled. Wells were replenished with fresh media to a final volume of 200 μl. The supernatants from triplicates for each culture condition were pooled. Using the Cytometric Bead Array kit (BD Biosciences Pharmingen), the presence of selected cytokines was quantified. Beads were analyzed used a FACScan (Becton Dickson Immunocytometry Systems).

Proliferation Assay. Approximately 0.05 to 0.1×10⁶ T cells were incubated with 0.3 to 0.6×10⁶ irradiated CD4-depleted PBMCs at 37° C. After approximately 72 to 96 hours of culture, cells were pulsed with 1 μCi of ³H-thymidine (MP Biomedicals, Irvine, Calif.). Cells were further incubated for 18 to 20 hours, and then harvested. Plates were harvested with a Packard Filtermate 196 cell harvester (Perkin Elmer, Shelton, Conn.), and filter-bound radioactivity was quantified using a Packard Microplate Scintillation Counter (Perkin Elmer). The average and standard error of the mean of triplicates for each culture condition was calculated.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents. All publications, patents and patent applications cited herein are incorporated by reference in their entirety into the disclosure.

What is claimed is:

1. An isolated population of regulatory T cells wherein the population comprises at least 75% regulatory T cells and less than 25% non-regulatory T cells, wherein the regulatory T cells:
(a) express CD4 and CD25; and
(b) do not express detectable levels of CD45RA and CD127.

2. The isolated population of regulatory T cells of claim 1, wherein the cells also express FoxP3.

3. The isolated population of regulatory T cells of claim 1, wherein the cells are isolated from peripheral blood mononuclear cells or synovial fluid.

4. The isolated population of regulatory T cells of claim 1, wherein the cells are isolated from tissue.

5. The isolated population of regulatory T cells of claim 4, wherein the tissue is selected from the group consisting of: spleen, thymus, lymph nodes, bone marrow, Peyer's patches, and tonsils.

6. An enriched population of regulatory T cells, wherein the regulatory T cells are:
(a) enriched at least two-fold compared to unfractionated cells;
(b) the cells express CD4 and CD25; and
(c) the cells do not express detectable levels of CD45RA and CD127.

7. The enriched population of claim 6, wherein the cells are enriched from a population of peripheral blood mononuclear cells or synovial fluid cells.

8. The enriched population of claim 6, wherein the cells are enriched from a tissue sample.

9. The enriched population of claim 8, wherein the tissue is selected from the group consisting of: spleen, thymus, lymph nodes, bone marrow, Peyer's patches, and tonsils.

10. The enriched population of claim 6, wherein the population is at least 5-fold enriched.

11. The enriched population of claim 10, wherein the population is at least 10-fold enriched.

12. The enriched population of claim 11, wherein the population is at least 50-fold enriched.

13. A method of enriching a population of regulatory T cells, comprising:
(a) contacting a population of cells with a first, second, third and fourth reagent, which respectively bind CD4, CD25, CD45RA and CD127; and
(b) selecting cells that bind to the first and second reagent and do not bind to the third or fourth reagent, wherein the selected cells are enriched for regulatory T cells.

14. The method of claim 13, wherein the first, second, third and fourth reagents comprise antibodies that respectively bind CD4, CD25, CD45RA and CD127.

15. The method of claim 14, wherein the antibodies are conjugated to a fluorochrome or magnetic particle.

16. The method of claim 13, wherein cell selection is performed by flow cytometry, fluorescence activated cell sorting, magnetic selection, affinity chromatography or panning.

17. The method of claim 16, wherein cell selection is performed by magnetic selection.

18. The method of claim 16, wherein cell selection is performed by fluorescence activated cell sorting.

19. A method of enriching a population of regulatory T cells, comprising:
(a) contacting a population of cells with a first, second, third and fourth reagent, which respectively bind CD4, CD25, CD45RO and CD127; and
(b) selecting cells that bind to the first, second and third reagent and do not bind to the fourth reagent, wherein the selected cells are enriched for regulatory T cells.

20. The method of claim 19, wherein the first, second, third and fourth reagents comprise antibodies that respectively bind CD4, CD25, CD45RO and CD127.

21. The method of claim 20, wherein the antibodies are conjugated to a fluorochrome or magnetic particle.

22. The method of claim 19, wherein cell selection is performed by flow cytometry, fluorescence activated cell sorting, magnetic selection, affinity chromatography or panning.

23. The method of claim 22, wherein cell selection is performed by magnetic selection.

24. The method of claim 22, wherein cell selection is performed by fluorescence activated cell sorting.

25. A method of enriching a population of regulatory T cells comprising:
(a) contacting a population of cells with antibodies that bind CD4 and CD25;
(b) retaining cells that bind to said antibodies that bind CD4 and CD25;
(c) contacting said retained cells with antibodies that bind CD45RA and CD127; and
(d) retaining cells that do not bind to said antibodies that bind CD45RA and CD127, wherein said retained cells are enriched for regulatory T cells.

26. The method of claim 25, wherein the population of cells are peripheral blood mononuclear cells or synovial fluid cells.

27. The method of claim 25, wherein the population of cells are from a tissue.

28. The method of claim 27, wherein the tissue is selected from the group consisting of: spleen, thymus, lymph nodes, bone marrow, Peyer's patches, and tonsils.

29. The method of claim 25, wherein said antibodies are conjugated to a fluorochrome or magnetic particle.

30. The method of claim 25, wherein said retaining is performed by flow cytometry, fluorescence activated cell sorting, magnetic selection, affinity chromatography or panning.

31. The method of claim 30, wherein said retaining is performed by magnetic selection.

32. The method of claim 30, wherein said retaining is performed by fluorescence activated cell sorting.

33. The method of claim 25, wherein said retaining is performed by magnetic selection and fluorescence activated cell sorting.

34. The method of claim 25, further comprising isolating the enriched population of regulatory T cells.

35. An enriched population of regulatory T cells isolated by the method of claim 34.

36. A method of enriching a population of regulatory T cells comprising:
(a) contacting a population of cells with antibodies that bind CD4, CD25 and CD45RO;
(b) retaining cells that bind to said antibodies that bind CD4, CD25 and CD45RO;
(c) contacting said retained cells with antibodies that bind CD127; and
(d) retaining cells that do not bind to said antibodies that bind CD127, wherein said retained cells are enriched for regulatory T cells.

37. The method of claim 36, wherein the population of cells are peripheral blood mononuclear cells or synovial fluid cells.

38. The method of claim 36, wherein the population of cells are from a tissue.

39. The method of claim 38, wherein the tissue is selected from the group consisting of: spleen, thymus, lymph nodes, bone marrow, Peyer's patches, and tonsils.

40. The method of claim 36, wherein said antibodies are conjugated to a fluorochrome or magnetic particle.

41. The method of claim 36, wherein said retaining is performed by flow cytometry, fluorescence activated cell sorting, magnetic selection, affinity chromatography or panning.

42. The method of claim 41, wherein said retaining is performed by magnetic selection.

43. The method of claim 41, wherein said retaining is performed by fluorescence activated cell sorting.

44. The method of claim 36, wherein said retaining is performed by magnetic selection and fluorescence activated cell sorting.

45. The method of claim 36, further comprising isolating the enriched population of regulatory T cells.

46. An enriched population of regulatory T cells isolated by the method of claim 45.

47. A method of suppressing an autoimmune response in a subject comprising:
(a) obtaining an enriched population of regulatory T cells, wherein the cells are obtained by:
(i) contacting a population of cells with a first, second, third and fourth reagent, which respectively bind CD4, CD25, CD45RA and CD127; and
(ii) selecting cells that bind to the first and second reagent and do not bind to the third or fourth reagent, wherein the selected cells are enriched for regulatory T cells; and
(b) introducing the enriched population of cells into the subject to suppress the autoimmune response.

48. The method of claim 47, wherein the enriched population of cells is obtained from the subject in need of treatment.

49. The method of claim 47, wherein the population of cells is enriched from peripheral blood mononuclear cells or synovial fluid.

50. The method of claim 47, wherein the population of cells is enriched from a tissue sample.

51. The method of claim 47, wherein said autoimmune response is associated with an autoimmune disease selected from the group consisting of: lupus erythematosus, pemphigus vulgaris, thyreoiditis, thrombocytopenic purpura, Graves disease, diabetes mellitus, myasthenia gravis, Addison's disease, rheumatoid arthritis, multiple sclerosis, psoriasis, uveitis, and autoimmune hemolytic anemia.

52. A method of suppressing an autoimmune response in a subject comprising:
(a) obtaining an enriched population of regulatory T cells, wherein the cells are obtained by:
(i) contacting a population of cells with a first, second, third and fourth reagent, which respectively bind CD4, CD25, CD45RO and CD127; and
(ii) selecting cells that bind to the first, second and third reagent and do not bind to the fourth reagent, wherein the selected cells are enriched for regulatory T cells; and
(b) introducing the enriched population of cells into the subject to suppress the autoimmune response.

53. The method of claim 52, wherein the enriched population of cells is obtained from the subject in need of treatment.

54. The method of claim 52, wherein the population of cells is enriched from peripheral blood mononuclear cells or synovial fluid.

55. The method of claim 52, wherein the population of cells is enriched from a tissue sample.

56. The method of claim 52, wherein said autoimmune response is associated with an autoimmune disease selected from the group consisting of: lupus erythematosus, pemphigus vulgaris, thyreoiditis, thrombocytopenic purpura, Graves disease, diabetes mellitus, myasthenia gravis, Addison's disease, rheumatoid arthritis, multiple sclerosis, psoriasis, uveitis, and autoimmune hemolytic anemia.

57. A method of enriching a population of regulatory T cells, comprising:
(a) contacting a population of cells with a first reagent or reagents which binds to a group of markers on non-CD4⁺ immune cells; and a second, third and fourth reagent, which respectively bind CD25, CD45RA and CD127; and
(b) selecting cells that bind to the second reagent and do not bind to the first, third or fourth reagent, wherein the selected cells are enriched for regulatory T cells.

58. The method of claim 57, wherein said non-CD4+ immune cells are selected from the group consisting of one or more of: cytotoxic T cells, γ/δ T cells, B cells, natural killer cells, dendritic cells, monocytes, granulocytes and erythroid cells.

59. The method of claim 58, wherein the markers on non-CD4⁺ immune cells are selected from the group consisting of one or more of: CD8, CD14, CD16, CD19, CD36, CD56, CD123, TCR γ/δ and glycophorin A.

60. The method of claim 57, wherein the first, second, third and fourth reagents are antibodies that respectively bind a group of markers on non-CD4⁺ immune cells, CD25, CD45RA and CD127.

61. The method of claim 60, wherein the antibodies are conjugated to a fluorochrome or magnetic particle.

62. The method of claim 57, wherein cell selection is performed by flow cytometry, fluorescence activated cell sorting, magnetic selection, affinity chromatography or panning.

63. The method of claim 62, wherein cell selection is performed by magnetic selection.

64. The method of claim 62, wherein cell selection is performed by fluorescence activated cell sorting.

65. A method of enriching a population of regulatory T cells, comprising:
(a) contacting a population of cells with a first reagent or reagents which binds to one or more of a group of markers on non-CD4⁺ immune cells; and a second, third and fourth reagent, which respectively bind CD25, CD45RO and CD127; and
(b) selecting cells that bind to the second and third reagent and do not bind to the first or fourth reagent, wherein the selected cells are enriched for regulatory T cells.

66. The method of claim 65, wherein said non-CD4⁺ immune cells are selected from the group consisting of one or more of: cytotoxic T cells, γ/δ T cells, B cells, natural killer cells, dendritic cells, monocytes, granulocytes and erythroid cells.

67. The method of claim 66, wherein the markers on non-CD4⁺ cells are selected from the group consisting of one or more of the following: CD8, CD14, CD16, CD19, CD36, CD56, CD123, TCR γ/δ and glycophorin A.

68. The method of claim 65, wherein the first, second, third and fourth reagents are antibodies that respectively bind a group of markers on non-CD4⁺ immune cells, CD25, CD45RA and CD127.

69. The method of claim 68, wherein the antibodies are conjugated to a fluorochrome or magnetic particle.

70. The method of claim 65, wherein cell selection is performed by flow cytometry, fluorescence activated cell sorting, magnetic selection, affinity chromatography or panning.

71. The method of claim 70, wherein cell selection is performed by magnetic selection.

72. The method of claim 70, wherein cell selection is performed by fluorescence activated cell sorting.

73. A method of enriching a population of regulatory T cells comprising:
(a) contacting a population of cells with antibodies that bind to one or more of a group of markers on non-CD4⁺ immune cells;
(b) retaining cells that do not bind to said antibodies that bind to one or more of a group of markers on non-CD4⁺ immune cells;
(c) contacting the retained cells with antibodies that bind CD25;
(d) retaining cells that bind to said antibodies that bind CD25;
(e) contacting said retained cells with antibodies that bind CD45RA and CD127; and
(f) retaining cells that do not bind to said antibodies that bind CD45RA and CD127, wherein said retained cells are enriched for regulatory T cells.

74. The method of claim 73, wherein said non-CD4⁺ immune cells are selected from the group consisting of one or more of: cytotoxic T cells, γ/δ T cells, B cells, natural killer cells, dendritic cells, monocytes, granulocytes and erythroid cells.

75. The method of claim 74, wherein the markers on non-CD4⁺ immune cells are selected from the group consisting of one or more of: CD8, CD14, CD16, CD19, CD36, CD56, CD123, TCR γ/δ and glycophorin A.

76. The method of claim 73, wherein the population of cells are peripheral blood mononuclear cells or synovial fluid cells.

77. The method of claim 73, wherein the population of cells are from a tissue.

78. The method of claim 77, wherein the tissue is selected from the group consisting of: spleen, thymus, lymph nodes, bone marrow, Peyer's patches, and tonsils.

79. The method of claim 73, wherein said antibodies are conjugated to a fluorochrome or magnetic particle.

80. The method of claim 73, wherein said retaining is performed by flow cytometry, fluorescence activated cell sorting, magnetic selection, affinity chromatography or panning.

81. The method of claim 80, wherein said retaining is performed by magnetic selection.

82. The method of claim 80, wherein said retaining is performed by fluorescence activated cell sorting.

83. The method of claim 73, wherein said retaining is performed by magnetic selection and fluorescence activated cell sorting.

84. The method of claim 73, further comprising isolating the enriched population of regulatory T cells.

85. An enriched population of regulatory T cells isolated by the method of claim 73.

86. A method of enriching a population of regulatory T cells comprising:
(a) contacting a population of cells with antibodies that bind to one or more of a group of markers on non-CD4⁺ immune cells;
(b) retaining cells that do not bind to said antibodies that bind to one or more or a group of markers on non-CD4⁺ immune cells;
(c) contacting the retained cells with antibodies that bind CD25;
(d) retaining cells that bind to said antibodies that bind CD25;
(e) contacting said retained cells with antibodies that bind CD45RO;
(f) retaining cells that bind to said antibodies that bind CD45RO;
(g) contacting the retained cells with antibodies that bind CD127;
(h) retaining cells that do not bind to said antibodies that bind CD127; wherein said retained cells are enriched for regulatory T cells.

87. The method of claim 86, wherein said non-CD4⁺ imnune cells are selected from the group consisting of one or more of: cytotoxic T cells, γ/δ T cells, B cells, natural killer cells, dendritic cells, monocytes, granulocytes and erythroid cells.

88. The method of claim 87, wherein the markers on non-CD4⁺ immune cells are selected from the group consisting of one or more of: CD8, CD14, CD16, CD19, CD36, CD56, CD123, TCRγ/δ and glycophorin A.

89. The method of claim 86, wherein the population of cells are peripheral blood mononuclear cells or synovial fluid cells.

90. The method of claim 86, wherein the population of cells are from a tissue.

91. The method of claim 90, wherein the tissue is selected from the group consisting of: spleen, thymus, lymph nodes, bone marrow, Peyer's patches, and tonsils.

92. The method of claim 86, wherein said antibodies are conjugated to a fluorochrome or magnetic particle.

93. The method of claim 86, wherein said retaining is performed by flow cytometry, fluorescence activated cell sorting, magnetic selection, affinity chromatography or panning.

94. The method of claim 93, wherein said retaining is performed by magnetic selection.

95. The method of claim 93, wherein said retaining is performed by fluorescence activated cell sorting.

96. The method of claim 86, wherein said retaining is performed by magnetic selection and fluorescence activated cell sorting.

97. The method of claim 86, further comprising isolating the enriched population of regulatory T cells.

98. An enriched population of regulatory T cells isolated by the method of claim 86.

99. A method of suppressing an autoimmune response in a subject comprising:
(a) obtaining an enriched population of regulatory T cells, wherein the cells are obtained by:
(i) contacting a population of cells with a first reagent or reagents which binds to one or more of a group of markers on non-CD4⁺ immune cells; and a second, third and fourth reagent which respectively bind CD25, CD45RA and CD127; and
(ii) selecting cells that bind to the second reagent and do not bind to the first, third or fourth reagent, wherein the selected cells are enriched for regulatory T cells; and
(b) introducing the enriched population of cells into the subject to suppress the autoimmune response.

100. The method of claim 99, wherein said non-CD4⁺ immune cells are selected from the group consisting of one or more of: cytotoxic T cells, γ/δ T cells, B cells, natural killer cells, dendritic cells, monocytes, granulocytes and erythroid cells.

101. The method of claim 100, wherein the markers on non-CD4⁺ immune cells are selected from the group consisting of one or more of: CD8, CD14, CD16, CD19, CD36, CD56, CD123, TCRγ/δ and glycophorin A.

102. The method of claim 99, wherein the enriched population of cells is obtained from the subject in need of treatment.

103. The method of claim 99, wherein the population of cells is enriched from peripheral blood mononuclear cells or synovial fluid.

104. The method of claim 99, wherein the population of cells is enriched from a tissue sample.

105. The method of claim 99, wherein said autoimmune response is associated with an autoimmune disease selected from the group consisting of: lupus erythematosus, pemphigus vulgaris, thyreoiditis, thrombocytopenic purpura, Graves disease, diabetes mellitus, myasthenia gravis, Addison's disease, rheumatoid arthritis, multiple sclerosis, psoriasis, uveitis, and autoimmune hemolytic anemia.

106. A method of suppressing an autoimmune response in a subject comprising:
(a) obtaining an enriched population of regulatory T cells, wherein the cells are obtained by:
(i) contacting a population of cells with a first reagent or reagents which binds to one or more of a group of markers on non-CD4⁺ immune cells; and a second, third and fourth reagent which respectively bind CD25, CD45RO and CD127; and
(ii) selecting cells that bind to the second and third reagent and do not bind to the first or fourth reagent, wherein the selected cells are enriched for regulatory T cells; and
(b) introducing the enriched population of cells into the subject to suppress the autoimmune response.

107. The method of claim 106, wherein said non-CD4⁺ immune cells are selected from the group consisting of one or more of: cytotoxic T cells, γ/δ T cells, B cells, natural killer cells, dendritic cells, monocytes, granulocytes and erythroid cells.

108. The method of claim 107, wherein the markers on non-CD4⁺ immune cells are selected from the group consisting of one or more of: CD8, CD14, CD16, CD19, CD36, CD56, CD123, TCR γ/δ and glycophorin A.

109. The method of claim 106, wherein the enriched population of cells is obtained from the subject in need of treatment.

110. The method of claim 106, wherein the population of cells is enriched from peripheral blood mononuclear cells or synovial fluid.

111. The method of claim 106, wherein the population of cells is enriched from a tissue sample.

112. The method of claim 106, wherein said autoimmune response is associated with an autoimmune disease selected from the group consisting of: lupus erythematosus, pemphigus vulgaris, thyreoiditis, thrombocytopenic purpura, Graves disease, diabetes mellitus, myasthenia gravis, Addison's disease, rheumatoid arthritis, multiple sclerosis, psoriasis, uveitis, and autoimmune hemolytic anemia.

113. A method of diagnosing an autoimmune disease comprising detecting a population of regulatory T cells, wherein the regulatory T cells:
(a) express CD4 and CD25; and
(b) do not express detectable levels of CD45RA and CD127.

114. An isolated population of regulatory T cells wherein the population comprises at least 75% regulatory T cells and less than 25% non-regulatory T cells, wherein the regulatory T cells:
(a) express CD4, CD25 and CD45RA; and
(b) do not express detectable levels of CD127.

115. The isolated population of regulatory T cells of claim 114, wherein the cells also express FoxP3.

116. The isolated population of regulatory T cells of claim 114, wherein the cells are isolated from peripheral blood mononuclear cells or synovial fluid.

117. The isolated population of regulatory T cells of claim 114, wherein the cells are isolated from tissue.

118. The isolated population of regulatory T cells of claim 117, wherein the tissue is selected from the group consisting of: spleen, thymus, lymph nodes, bone marrow, Peyer's patches, and tonsils.

119. An enriched population of regulatory T cells, wherein the regulatory T cells are:
(a) enriched at least two-fold compared to unfractionated cells;
(b) the cells express CD4, CD25 and CD45RA; and
(c) the cells do not express detectable levels of CD127.

120. The enriched population of claim 119, wherein the cells are enriched from a population of peripheral blood mononuclear cells or synovial fluid cells.

121. The enriched population of claim 119, wherein the cells are enriched from a tissue sample.

122. The enriched population of claim 121, wherein the tissue is selected from the group consisting of: spleen, thymus, lymph nodes, bone marrow, Peyer's patches, and tonsils.

123. The enriched population of claim 119, wherein the population is at least 5-fold enriched.

124. The enriched population of claim 123, wherein the population is at least 10-fold enriched.

125. The enriched population of claim 124, wherein the population is at least 50-fold enriched.

126. A method of enriching a population of regulatory T cells, comprising:
(a) contacting a population of cells with a first, second, third and fourth reagent, which respectively bind CD4, CD25, CD45RA and CD127; and
(b) selecting cells that bind to the first, second and third reagent and do not bind to the fourth reagent, wherein the selected cells are enriched for regulatory T cells.

127. The method of claim 126, wherein the first, second, third and fourth reagents comprise antibodies that respectively bind CD4, CD25, CD45RA and CD127.

128. The method of claim 127, wherein the antibodies are conjugated to a fluorochrome or magnetic particle.

129. The method of claim 126, wherein cell selection is performed by flow cytometry, fluorescence activated cell sorting, magnetic selection, affinity chromatography or panning.

130. The method of claim 129, wherein cell selection is performed by magnetic selection.

131. The method of claim 129, wherein cell selection is performed by fluorescence activated cell sorting.

132. A method of enriching a population of regulatory T cells comprising:
(a) contacting a population of cells with antibodies that bind CD4, CD25 and CD45RA;
(b) retaining cells that bind to said antibodies that bind CD4, CD25 and CD45RA;
(c) contacting said retained cells with antibodies that bind CD127; and
(d) retaining cells that do not bind to said antibodies that bind CD127, wherein said retained cells are enriched for regulatory T cells.

133. The method of claim 132, wherein the population of cells are peripheral blood mononuclear cells or synovial fluid cells.

134. The method of claim 132, wherein the population of cells are from a tissue.

135. The method of claim 134, wherein the tissue is selected from the group consisting of: spleen, thymus, lymph nodes, bone marrow, Peyer's patches, and tonsils.

136. The method of claim 132, wherein said antibodies are conjugated to a fluorochrome or magnetic particle.

137. The method of claim 132, wherein said retaining is performed by flow cytometry, fluorescence activated cell sorting, magnetic selection, affinity chromatography or panning.

138. The method of claim 137, wherein said retaining is performed by magnetic selection.

139. The method of claim 137, wherein said retaining is performed by fluorescence activated cell sorting.

140. The method of claim 132, wherein said retaining is performed by magnetic selection and fluorescence activated cell sorting.

141. The method of claim 132, further comprising isolating the enriched population of regulatory T cells.

142. An enriched population of regulatory T cells isolated by the method of claim 141.

143. A method of suppressing an autoimmune response in a subject comprising:
(a) obtaining an enriched population of regulatory T cells, wherein the cells are obtained by:
(i) contacting a population of cells with a first, second, third and fourth reagent, which respectively bind CD4, CD25, CD45RA and CD127; and
(ii) selecting cells that bind to the first, second and third reagent and do not bind to the fourth reagent, wherein the selected cells are enriched for regulatory T cells; and
(b) introducing the enriched population of cells into the subject to suppress the autoimmune response.

144. The method of claim 143, wherein the enriched population of cells is obtained from the subject in need of treatment.

145. The method of claim 143, wherein the population of cells is enriched from peripheral blood mononuclear cells or synovial fluid.

146. The method of claim 143, wherein the population of cells is enriched from a tissue sample.

147. The method of claim 143, wherein said autoimmune response is associated with an autoimmune disease selected from the group consisting of: lupus erythematosus, pemphigus vulgaris, thyreoiditis, thrombocytopenic purpura, Graves disease, diabetes mellitus, myasthenia gravis, Addison's disease, rheumatoid arthritis, multiple sclerosis, psoriasis, uveitis, and autoimmune hemolytic anemia.

148. A method of enriching a population of regulatory T cells, comprising:
(a) contacting a population of cells with a first reagent or reagents which binds to a group of markers on non-CD4⁺ immune cells; and a second, third and fourth reagent, which respectively bind CD25, CD45RA and CD127; and
(b) selecting cells that bind to the second and third reagent and do not bind to the first or fourth reagent, wherein the selected cells are enriched for regulatory T cells.

149. The method of claim 148, wherein said non-CD4⁺ immune cells are selected from the group consisting of one or more of: cytotoxic T cells, γ/δ T cells, B cells, natural killer cells, dendritic cells, monocytes, granulocytes and erythroid cells.

150. The method of claim 149, wherein the markers on non-CD4⁺ immune cells are selected from the group consisting of one or more of: CD8, CD14, CD16, CD19, CD36, CD56, CD123, TCR γ/δ and glycophorin A.

151. The method of claim 148, wherein the first, second, third and fourth reagents are antibodies that respectively bind a group of markers on non-CD4⁺ immune cells, CD25, CD45RA and CD127.

152. The method of claim 151, wherein the antibodies are conjugated to a fluorochrome or magnetic particle.

153. The method of claim 148, wherein cell selection is performed by flow cytometry, fluorescence activated cell sorting, magnetic selection, affinity chromatography or panning.

154. The method of claim 153, wherein cell selection is performed by magnetic selection.

155. The method of claim 153, wherein cell selection is performed by fluorescence activated cell sorting.

156. A method of enriching a population of regulatory T cells comprising:
(a) contacting a population of cells with antibodies that bind to one or more of a group of markers on non-CD4⁺ immune cells;
(b) retaining cells that do not bind to said antibodies that bind to one or more of a group of markers on non-CD4⁺ immune cells;
(c) contacting the retained cells with antibodies that bind CD25 and CD45RA;
(d) retaining cells that bind to said antibodies that bind CD25 and CD45RA;
(e) contacting said retained cells with antibodies that bind CD127; and
(f) retaining cells that do not bind to said antibodies that bind CD127, wherein said retained cells are enriched for regulatory T cells.

157. The method of claim 156, wherein said non-CD4⁺ immune cells are selected from the group consisting of one or more of: cytotoxic T cells, γ/δ T cells, B cells, natural killer cells, dendritic cells, monocytes, granulocytes and erythroid cells.

158. The method of claim 157, wherein the markers on non-CD4⁺ immune cells are selected from the group consisting of one or more of: CD8, CD14, CD16, CD19, CD36, CD56, CD123, TCR γ/δ and glycophorin A.

159. The method of claim 156, wherein the population of cells are peripheral blood mononuclear cells or synovial fluid cells.

160. The method of claim 156, wherein the population of cells are from a tissue.

161. The method of claim 160, wherein the tissue is selected from the group consisting of: spleen, thymus, lymph nodes, bone marrow, Peyer's patches, and tonsils.

162. The method of claim 156, wherein said antibodies are conjugated to a fluorochrome or magnetic particle.

163. The method of claim 156, wherein said retaining is performed by flow cytometry, fluorescence activated cell sorting, magnetic selection, affinity chromatography or panning.

164. The method of claim 163, wherein said retaining is performed by magnetic selection.

165. The method of claim 163, wherein said retaining is performed by fluorescence activated cell sorting.

166. The method of claim 156, wherein said retaining is performed by magnetic selection and fluorescence activated cell sorting.

167. The method of claim 156, further comprising isolating the enriched population of regulatory T cells.

168. An enriched population of regulatory T cells isolated by the method of claim 156.

169. A method of suppressing an autoimmune response in a subject comprising:
(a) obtaining an enriched population of regulatory T cells, wherein the cells are obtained by:
(i) contacting a population of cells with a first reagent or reagents which binds to one or more of a group of markers on non-CD4⁺ immune cells; and a second, third and fourth reagent which respectively bind CD25, CD45RA and CD127; and
(ii) selecting cells that bind to the second and third reagent and do not bind to the first or fourth reagent, wherein the selected cells are enriched for regulatory T cells; and
(b) introducing the enriched population of cells into the subject to suppress the autoimmune response.

170. The method of claim 169, wherein said non-CD4⁺ immune cells are selected from the group consisting of one or more of: cytotoxic T cells, γ/δ T cells, B cells, natural killer cells, dendritic cells, monocytes, granulocytes and erythroid cells.

171. The method of claim 170, wherein the markers on non-CD4⁺ immune cells are selected from the group consisting of one or more of: CD8, CD14, CD16, CD19, CD36, CD56, CD123, TCR γ/δ and glycophorin A.

172. The method of claim 169, wherein the enriched population of cells is obtained from the subject in need of treatment.

173. The method of claim 169, wherein the population of cells is enriched from peripheral blood mononuclear cells or synovial fluid.

174. The method of claim 169, wherein the population of cells is enriched from a tissue sample.

175. The method of claim 169, wherein said autoimmune response is associated with an autoimmune disease selected from the group consisting of: lupus erythematosus, pemphigus vulgaris, thyreoiditis, thrombocytopenic purpura, Graves disease, diabetes mellitus, myasthenia gravis, Addison's disease, rheumatoid arthritis, multiple sclerosis, psoriasis, uveitis, and autoimmune hemolytic anemia.
